SEQUENCE LISTING (12 sequences)

SEQ ID NO: 1 - protein, 6 aa, synthetic construct
SDYAWN

SEQ ID NO: 2 - protein, 16 aa, synthetic construct
YISYSGSTTYNPSLKS

SEQ ID NO: 3 - protein, 8 aa, synthetic construct
GYYYGSGF

SEQ ID NO: 4 - protein, 15 aa, synthetic construct
KASESVEYFGTSLMH

SEQ ID NO: 5 - protein, 7 aa, synthetic construct
AASNRES

SEQ ID NO: 6 - protein, 9 aa, synthetic construct
QQTRKVPYT

SEQ ID NO: 7 - protein, 117 aa, synthetic construct
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSS

SEQ ID NO: 8 - protein, 111 aa, synthetic construct
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIK

SEQ ID NO: 9 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 447 aa, synthetic construct
QVQLQESGPGLVKPSDTLSLTCAVSGNSITSDYAWNWIRQPPGKGLEWIGYISYSGSTTYNPSLKSRVTMSRDTSKNQFSLKLSSVTAVDTAVYYCATGYYYGSGFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 218 aa, synthetic construct
DIVLTQSPDSLAVSLGERATINCKASESVEYFGTSLMHWYQQKPGQPPKLLIYAASNRESGVPDRFSGSGSGTDFTLTISSLQAEDVAVYYCQQTRKVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0216384 A1
(43) Pub. Date: Jul. 30, 2026

(54) COMPOSITIONS AND METHODS FOR THE TREATMENT OF PROSTATE CANCER

(71) Applicant: Janssen Biotech, Inc., Horsham, PA (US)

(72) Inventors: Edward Fathi Attiyeh, Raritan, NJ (US); Charles George DRAKE, Raritan, NJ (US); Jeffrey Robert INFANTE, Raritan, NJ (US); Won KIM, Raritan, NJ (US); Josh David LAURING, Raritan, NJ (US); Steven Irwin MAX, Raritan, NJ (US)

(21) Appl. No.: 19/211,544

(22) Filed: May 19, 2025

Related U.S. Application Data
(60) Provisional application No. 63/649,683, filed on May 20, 2024.
(60) Provisional application No. 63/735,508, filed on Dec. 18, 2024.
(60) Provisional application No. 63/746,530, filed on Jan. 17, 2025.
(60) Provisional application No. 63/759,862, filed on Feb. 18, 2025.

(51) Int. Cl. A61K 51/10; A61P 35/00; A61P 35/04; G01N 33/57555

(52) U.S. Cl. CPC A61K 51/1075; A61K 51/1093; A61P 35/00; A61P 35/04; G01N 33/57555; G01N 2333/96433

(57) ABSTRACT

Compositions and methods for the treatment of cancer, in particular prostate cancer, are described. According to certain embodiments, a method of treating cancer in a patient comprises administering to the patient a therapeutically effective amount of a radioconjugate, wherein the radioconjugate comprises an antibody or antigen binding domain with binding specificity for hK2.

### CROSS-REFERENCE TO RELATED APPLICATIONS

This application claims priority to U.S. Provisional Application No. 63/649,683, filed on May 20, 2024, U.S. Provisional Application No. 63/735,508, filed on Dec. 18, 2024, U.S. Provisional Application No. 63/746,530, filed on Jan. 17, 2025, and U.S. Provisional Application No. 63/759,862, filed on Feb. 18, 2025, the disclosures of each of which are hereby incorporated by reference in their entireties.

### REFERENCE TO SEQUENCE LISTING SUBMITTED ELECTRONICALLY

This application contains a sequence listing, which is submitted electronically in XML format with a file name of “065768.179US5” and a creation date of May 16, 2025, and having a size of 12,696 bytes. The sequence listing is part of the specification and is herein incorporated by reference in its entirety.

### TECHNICAL FIELD

Embodiments of the present invention relate to compositions and methods for the treatment of prostate cancer. In particular, embodiments of the present invention relate to radioconjugate compositions for hK2-targeted therapies.

### BACKGROUND

Prostate cancer is one of the most common forms of cancer. The growth of the tumor is usually a process that takes place over a long period of time. Prostate cancer is often a mild form of cancer. In fact, the majority of people diagnosed with prostate cancer survive and recover. A minority of people encounter a more aggressive form of prostate cancer, which metastasizes in an early stage. This aggressive form of prostate cancer can only be curable if it is diagnosed at an early stage, before the cancer has spread to extracapsular tissue.

The landscape for the management of patients with metastatic castration-resistant prostate cancer (mCRPC) has changed with the approval of several new agents, including androgen receptor-(AR) directed therapy (e.g., enzalutamide and abiraterone acetate plus prednisone), chemotherapy (e.g., docetaxel and cabazitaxel), and cellular immune therapy (e.g., Sipuleucel-T). With these agents, overall survival has improved from the previously reported range of 6 to 10 months to 18 to 24 months. However, most prostate cancer patients will experience disease progression on anti-androgen or androgen synthesis inhibitor therapy within 13 to 20 months.

There remains a need for new therapeutic agents and methods for treating and diagnosing prostate cancer; in particular, therapies with mechanisms of action that overcome pathways of resistance are crucial in developing alternative strategies for the treatment of mCRPC.

### SUMMARY

The application describes a method of treating cancer in a patient comprises administering to the patient a therapeutically effective amount of a radioconjugate, wherein the radioconjugate comprises an antibody or antigen binding domain with binding specificity for hK2.

Accordingly, in a general aspect the present application provides a method of treating prostate cancer, preferably advanced prostate cancer, preferably metastatic castration-resistant prostate cancer (mCRPC), in a patient in need thereof, comprising:
i) administering to the patient a (therapeutically effective) first dose of a radioconjugate comprising an antibody or an antigen binding fragment having binding specificity for hK2 conjugated to a chelator, optionally via a linker, wherein actinium-225 (225Ac) is chelated to the chelator; wherein no prostate-specific antigen (PSA) progression relative to a serum PSA level of the patient measured prior to the administering of (i), preferably relative prior to any administering of the radioconjugate (pretreatment baseline level), is observed for a period of time after the administering of the first dose, preferably for a period of at least 12 weeks after the administering of the first dose; and
ii) administering to the patient a (therapeutically effective) second dose of the radioconjugate after the period of time if (i), if the following conditions are met (before the administering of the second dose):
   a. there is a PSA progression after the period of time of (i) relative to the lowest serum PSA level of the patient (nadir) from immediately prior to the administering of (i) to the end of the period of time; and
   b. the patient has a platelet count above 100×10E9 per liter of blood;
iii) optionally repeating (ii).

In some embodiments, during the period of time of (i), there is no administering of another dose of the radioconjugate (other than the first dose of (i)).

In some embodiments, the period of time in (i) is weeks or more than 12 weeks, such as 12-96 weeks, or 12-80 weeks, or 12-60 weeks, or 12-48 weeks, or 12-40 weeks, for example 12 or 38 weeks.

In some embodiments, the method further comprising monitoring the serum PSA level of the patient after the administering of step (i), such as measuring or obtaining a measurement of the serum PSA level of the patient one or more times after the administering of step (i), for example, the serum PSA level is measured once every 4-6 weeks, preferably once every 4 weeks, such as three times every 4 weeks, and optionally further comprising measuring or obtaining the measurement of the serum PSA level of the patient immediately before the administering of (i).

In some embodiments, the second dose of the radioconjugate of (ii) is not administered to the patient, if there is no PSA progression relative to the lowest serum PSA level (nadir) of the patient, and/or if the patient has a platelet count of 100×10E9 per liter of blood or lower.

In some embodiments, there has been no PSA progression in (i), when any serum PSA level measured during the period is not 25% or more higher than the serum PSA level of the patient measured prior to (i), preferably prior to any administering of the radioconjugate (pretreatment baseline level).

In some embodiments, there has been no PSA progression in (i), when any serum PSA level measured during the period is not 2 ng/mL or more higher than the serum PSA level of the patient measured prior to (i), preferably prior to any administering of the radioconjugate (pretreatment baseline level).

In some embodiments, there has been no PSA progression in (i), when any serum PSA level measured during the period is not 25% or more and not 2 ng/ml or more higher than the serum PSA level of the patient measured prior to step (i), preferably prior to any administering of the radioconjugate (pretreatment baseline level).

In some embodiments, there is a PSA progression in (ii)a, when a serum PSA level measured after the period is 25% or more higher than the lowest serum PSA level of the patient measured over the period of (i) or immediately prior to the administering of step (i).

In some embodiments, there is a PSA progression in (ii)a, when a serum PSA level measured after the period is 2 ng/ml or more higher than the lowest serum PSA level of the patient measured over the period of (i) or immediately prior to the administering of step (i).

In some embodiments, there is a PSA progression in (ii)a, when a serum PSA level measured after the period is 25% or more and 2 ng/ml or more higher than the lowest serum PSA level of the patient measured over the period of (i) or immediately prior to the administering of step (i).

In some embodiments, the dose of the radioconjugate administered in step (i) is 100-400 μCi, such as a dose of 150 μCi, 200 μCi, 250 μCi, 300 μCi or 400 μCi, preferably 150-300 μCi, preferably 200-250 μCi, such as 200 μCi or 250 μCi, preferably 250 μCi.

In some embodiments, the first dose of the radioconjugate administered in (i) is 150 μCi, 200 μCi or 250 μCi.

In some embodiments, the first dose of the radioconjugate administered in (i) is 200 μCi or 250 μCi, preferably 250 μCi.

In some embodiments, the first dose of the radioconjugate administered in (i) is 150 μCi or 200 μCi, preferably 150 μCi.

In some embodiments, the patient has received one or more doses of the radioconjugate prior to step (i).

In some embodiments, the patient has not received any dose of the radioconjugate prior to step (i).

In some embodiments, the second dose of the radioconjugate administered in step (ii) is 100-250 μCi, such as 100 μCi, 150 μCi, 200 μCi, or 250 μCi, preferably 100 or 150 μCi.

In some embodiments, the second dose of the radioconjugate administered in step (ii) is 100 or 150 μCi.

In some embodiments, the patient has not received any dose of the radioconjugate prior to step (i), wherein the first dose of the radioconjugate administered in step (i) is 200 μCi or 250 μCi, preferably 250 μCi, and wherein the second dose of the radioconjugate administered in step (ii) is 100 or 150 μCi.

In some embodiments, the patient has not received any dose of the radioconjugate prior to step (i), wherein the first dose of the radioconjugate administered in step (i) is 150 μCi or 200 μCi, preferably 150 μCi, and wherein the second dose of the radioconjugate administered in step (ii) is 100 or 150 μCi.

In some embodiments, the second dose of the radioconjugate administered in step (ii) is 150 μCi, if cumulative exposure to the radioconjugate for the patient prior to step (ii) is preferably not higher than 500 μCi, or is not higher than 450 μCi.

In some embodiments, the second dose of the radioconjugate administered in step (ii) is 100 μCi, if cumulative exposure to the radioconjugate for the patient prior to step (ii) is 500 μCi.

In certain embodiments, the second dose of the radioconjugate administered in step (ii) is lower than the dose of the radioconjugate administered in step (i).

In some embodiments, the method further comprises (iii) administering to the patient a third dose of the radioconjugate after a (second) period of time after (ii), preferably at least 12 weeks after (ii), if the following conditions are met: a) there is a PSA progression after the (second) period, relative to the lowest serum PSA level of the patient (nadir) measured over the (second) period or immediately prior to the administering of (ii); and b) the patient has a platelet count above 100×10E9 per liter of blood.

In some embodiments, the cumulative exposure to the radioconjugate does not exceed 600 μCi, preferably does not exceed 500 μCi.

In some embodiments, the antibody or an antigen binding fragment comprises a heavy chain complementarity determining region (HCDR) 1, a HCDR2 and a HCDR3 of a heavy chain variable region (VH) comprising the amino acid sequence of SEQ ID NO: 7, and a light chain complementarity determining region (LCDR) 1, a LCDR2, and a LCDR3 of a light chain variable region (VL) comprising the amino acid sequence of SEQ ID NO: 8, wherein the HCDR1, HCDR2, HCDR3, LCDR1, LCDR2 and LCDR3 are defined by the Kabat, Chothia, IMGT or AbM numbering system.

In certain embodiments, the antibody or an antigen binding fragment comprises the HCDR1, HCDR2, HCDR3, LCDR1, LCDR2 and LCDR3 having the amino acid sequences of SEQ ID NOs: 1, 2, 3, 4, 5, and 6, respectively.

In certain embodiments, the antibody or an antigen binding fragment comprises the VH and VL having the amino acid sequences at least 90% identical to SEQ ID NO: 7 and SEQ ID NO: 8, respectively.

In certain embodiments, the antibody comprises a heavy chain sequence and a light chain sequence having the amino acid sequences at least 90% identical to SEQ ID NO: 11 and SEQ ID NO: 12, respectively.

In some embodiments, the chelator is 1,4,7,10-tetraazacyclododecane-1,4,7,10,tetraacetic acid (DOTA).

In some embodiments, the linker comprises or is a benzyl isothiocyanate, such as 4-isothiocyanatobenzyl, 3-isothiocyanatobenzyl, 2-isothiocyanatobenzyl, wherein the benzyl isothiocyanate optionally has one or more alkyl substituents and/or one or more cyclic acid substituents, e.g., wherein the benzyl isothiocyanate optionally has substituents L1 and/or L2 at the benzylic position, wherein:
L1 is H or an alkyl or cyclic alkyl (when L1 is an alkyl or cyclic alkyl, the alkyl or cyclic alkyl can have carbon or heteroatom substitutions);
L2 is H or an alkyl or cyclic alkyl. (when L1 is an alkyl or cyclic alkyl, the alkyl or cyclic alkyl can have carbon or heteroatom substitutions); or
L1 and L2 form a cycle, such as a carbon cycle or a heterocycle or a fused cycle. In some embodiments, the antibody or antigen binding fragment is conjugated to the chelator via a linker.

In some embodiments, the radioconjugate comprises the antibody or antigen binding fragment conjugated to 2-(4-isothiocyanatobenzyl)-1,4,7,10-tetraazacyclododecane-1,4,7,10-tetraacetic acid with the chelated actinium-225 (²²⁵Ac), preferably, the 2-(4-isothiocyanatobenzyl)-1,4,7,10-tetraazacyclododecane-1,4,7,10-tetraacetic acid is covalently linked to one or more amino acid residues in the Fc region of the antibody.

In some embodiments, the radioconjugate comprises the antibody or antigen binding fragment conjugated to 2-S-(4-isothiocyanatobenzyl)-1,4,7,10-tetraazacyclododecane-1,4,7,10-tetraacetic acid with the chelated actinium-225 (225Ac), preferably, the 2-S-(4-isothiocyanatobenzyl)-1,4,7,10-tetraazacyclododecane-1,4,7,10-tetraacetic acid is covalently linked to one or more amino acid residues in the Fc region of the antibody.

In some embodiments, the radioconjugate comprises an average of from about 1 to about 4 chelator molecules conjugated to the antibody.

In some embodiments, the radioconjugate provides a specific activity from about 50 μCi to about 350 μCi, such as 50 μCi, 100 μCi, 150 μCi, 200 μCi, per about 2-3 mg of total antibody.

In some embodiments, the radioconjugate is administered in a pharmaceutical composition, preferably in a pharmaceutical composition formulated for intravenous administration, wherein the pharmaceutical composition comprises the radioconjugate and one or more pharmaceutically acceptable excipients.

In some embodiments, the pharmaceutical composition comprises the radioconjugate, a radioprotectant, surfactant and buffer, with a pH of 5-6.

In certain embodiments, the pharmaceutical composition further comprises a conjugate comprising the antibody or the antigen binding fragment conjugated to the chelator without actinium-225 (²²⁵Ac) chelated by the chelator.

In certain embodiments, the pharmaceutical composition is administered with a total antibody mass of 2 to 10 mg per dose.

In certain embodiments, the pharmaceutical composition is administered at 12.5 μCi/mL, 25 μCi/mL, 37.5 μCi/mL or 50 μCi/mL (at the time of dosing).

In some embodiments, the radioconjugate or the pharmaceutical composition is administered to the patient intravenously.

In some embodiments, the patient:
1) has a prior treatment with at least one androgen receptor pathway inhibitor (ARPI);
2) can have a prior chemotherapy treatment;
3) has not had prior radioconjugate therapy; and/or
4) has no prior radiotherapy with >30% red marrow distribution.

In some embodiments, the patient has received 0 to 12, for example 4, prior lines of therapy, such as ARPI and/or taxane.

In some embodiments, the patient has met one or more, such as all, of the following criteria:
1) mCRPC with histologic confirmation of adenocarcinoma;
2) prior exposure to at least one androgen receptor-targeted therapy (eg, abiraterone acetate, enzalutamide, apalutamide, darolutamide), or prior taxane or other chemotherapy;
3) treatment with other agents for prostate cancer, if received, discontinued ≥2 weeks prior to first dose of the radioconjugate;
4) prior orchiectomy or medical castration; or, for patients who have not undergone orchiectomy, receiving ongoing androgen deprivation therapy with a gonadotropin releasing hormone (GnRH) analog (agonist or antagonist) prior to the first dose of the radioconjugate;
5) palliative radiotherapy (eg, soft tissue lesions) completed >2 weeks prior to the first dose of the radioconjugate, except for palliative radiotherapy for pain (eg, bone pain), which may be used any time prior to first dose;
6) Eastern Cooperative Oncology Group (ECOG) performance status of 0 or 1;
7) Hematology laboratory parameters within the following range:
   a. Hemoglobin >9.0 g/dL
   b. Absolute neutrophil count >1.5×10⁹/L
   c. Platelet count >100×10⁹/L
8) Clinical chemistry laboratory parameters within the following range before the first dose of study drug:
   a. Serum total bilirubin <1.5×upper limit of normal (ULN)
   b. Aspartate aminotransferase (AST) and alanine aminotransferase (ALT)≤2.5×ULN (or ≤4×ULN for participants with tumor involvement in the liver)
   c. Calculated or measured creatinine clearance >50 mL/min/1.73 m² as determined by Modification of Diet in Renal Disease formula
   d. Grade ≤1 proteinuria. For proteinuria clearly attributable to other known medical conditions of the participant, such as hemorrhagic radiation cystitis or chronic hematuria caused by tumor infiltrates, Grade ≥1 proteinuria requires an approval from the sponsor's medical monitor.

In some embodiments, the patient does not have one or more of the following:
1) Prior treatment with radium Xofigo® (Ra 223 dichloride), strontium, or samarium therapy or radioconjugate therapy;
2) Prior radiation therapy encompassing >30% of expected red marrow distribution;
3) Diffuse bone or bone-marrow involvement (ie, superscan—diffuse increased marrow uptake with reduced background and renal activity);
4) Spinal cord compression (unless treated, stable, and approved by the sponsor medical monitor);
5) Active CNS metastases;
6) Known history of myelodysplastic syndrome, leukemia, or hematological malignancy with features suggestive of myelodysplastic syndrome/acute myeloid leukemia at any timepoint;
7) Toxicity from prior anticancer therapy has not resolved to baseline levels or to Grade ≤1 (except alopecia, radiation tissue fibrosis, or peripheral neuropathy);
8) Malignancy diagnosis other than the disease under study within 2 years prior to the first dose of study drug, except for squamous and basal cell carcinoma of the skin, non-muscle invasive bladder cancer, or any malignancy considered cured or has minimal risk of recurrence within 1 year of first dose of study drug (in the opinion of both the investigator and sponsor's medical monitor);
9) Any systemic anti-neoplastic therapy (eg, chemotherapy, immunotherapy or biological therapy [including mAbs], poly ADP ribose polymerase [PARP] inhibitors)≤30 days prior to the first dose of the radioconjugate except for luteinizing hormone-releasing hormone agonists/antagonists or GnRH agonists/antagonists, and androgen axis drugs (e.g., enzalutamide, apalutamide, darolutamide, and/or abiraterone acetate plus prednisone) must have been discontinued ≥2 weeks prior to the first dose of the radioconjugate.

In some embodiments, the treatment provides a lower rate of severe toxicities (toxicity of Grade 3 or higher according to the NCI CTCAE version 5.0), e.g., compared to the administering of the first dose of (i) at a fixed dose level and at a fixed dose schedule (e.g., compared to the administering of the first dose of (i) once every 8 weeks).

In some embodiments, the treatment results in effective treatment of the prostate cancer, as measured by the PSA response, and/or Overall response rate (ORR) according to response criteria of Prostate Cancer Working Group 3 (PCWG3).

In some embodiments, the prostate cancer is advanced prostate cancer, locally advanced prostate cancer, metastatic castration-sensitive prostate cancer (CSPC), metastatic castration-resistant prostate cancer (CRPC), non-metastatic CSPC, non-metastatic CRPC, or metastatic prostate cancer, preferably the prostate cancer is metastatic CRPC.

In some embodiments, the prostate cancer is metastatic hormone sensitive prostate cancer. In some embodiments, the prostate cancer is metastatic hormone sensitive prostate cancer with non-castrate levels of testosterone, for example with levels of testosterone >150 ng/dL.

In another general aspect the present application provides the radioconjugate, the pharmaceutical composition, and a product comprising the radioconjugate as defined in any one of the foregoing embodiments for use in the method of any one of the preceding embodiments.

Further aspects, features and advantages of the present invention will be better appreciated upon a reading of the following detailed description of the invention and claims.

### BRIEF DESCRIPTION OF THE DRAWINGS

The following drawings form part of the present specification and are included to further demonstrate certain aspects of the present invention. The invention can be better understood by reference to one or more of these drawings in combination with the description of specific embodiments presented herein.

The patent or application file contains at least one drawing executed in color. Copies of this patent or patent application publication with color drawing(s) will be provided by the Office upon request and payment of the necessary fee.

FIG. 1 is a spider plot of percent changes from baseline in the PSA level over time for subjects in the 250 μCi escalation cohort who received a single dose treatment (all treated analysis set).

FIG. 2 illustrates the PSA level of a patient treated with a method according to an embodiment of the application.

### DETAILED DESCRIPTION OF THE INVENTION

The disclosed methods can be understood more readily by reference to the following detailed description taken in connection with the accompanying figures, which form a part of this disclosure. It is to be understood that the disclosed methods are not limited to the specific methods described and/or shown herein, and that the terminology used herein is for the purpose of describing particular embodiments by way of example only and is not intended to be limiting of the claimed methods. All patents, published patent applications, and publications cited herein are incorporated by reference as if set forth fully herein.

Various terms relating to aspects of the description are used throughout the specification and claims. Such terms are to be given their ordinary meaning in the art unless otherwise indicated. Other specifically defined terms are to be construed in a manner consistent with the definitions provided herein.

As used in this specification and the appended claims, the singular forms “a,” “an,” and “the” include plural referents unless the content clearly dictates otherwise. Thus, for example, reference to “a cell” includes a combination of two or more cells, and the like.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

The transitional terms “comprising,” “consisting essentially of,” and “consisting of” are intended to connote their generally accepted meanings in the patent vemacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The compounds, compositions and methods described herein are useful in treating cancer in a patient in need of such treatment. Embodiments of the compounds, compositions and methods are effective in treating prostate cancer, including advanced stages of prostate cancer, particularly castration-resistant prostate cancer (CRPC) and, thus, result in longer survival rates for patients. These compounds are particularly useful in patients where existing treatments for advanced stages of prostate are deemed unsuccessful.

Human kallikrein 2 (hK2) is a trypsin-like antigen produced by columnar prostate epithelial cells and driven by androgen receptor (AR) signaling in a manner identical to the closely related prostate-specific antigen (PSA; human glandular kallikrein 3) with which genetically it has an 80% homology with the PSA gene. Unlike PSA however, circulating levels of hK2 are found at exceptionally low levels, where they can be bound by multiple protease inhibitor complexes. Although hK2 is believed to be primarily secreted, there is evidence that it is capable of inducing internalization via an antigen-antibody complex hence it is believed to exist on the cell surface as well. Because hK2 expression is highly specific for prostate adenocarcinoma and increases throughout disease progression, hK2-targeted therapies are attractive.

An exemplary hK2 amino acid sequence is described as Transcript: KLK2-201 (ENST00000325321), a product of gene ENSG00000167751, as given in the ensemble database.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions can be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins can be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species can be assigned to one of two clearly distinct types, namely kappa (κ) and lambda (λ), based on the amino acid sequences of their constant domains.

“Antigen binding fragment” or “antigen binding domain” refers to a portion of an isolated protein that binds an antigen. Antigen binding fragments can be synthetic, enzymatically obtainable or genetically engineered polypeptides and include portions of an immunoglobulin that bind an antigen, such as the VH, the VL, the VH and the VL, Fab, Fab′, F(ab′)2, Fd and Fv fragments, domain antibodies (dAb) consisting of one VH domain or one VL domain, shark variable IgNAR domains, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as VH and VL) can be linked together via a synthetic linker to form various types of single antibody designs where the VH/VL domains can pair intramolecularly, or intermolecularly in those cases when the VH and VL domains are expressed by separate single chains, to form a monovalent antigen binding domain, such as single chain Fv (scFv) or diabody.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs can be defined using various delineations such as Kabat (Wu et al. (1970) J Exp Med 132:211-50; Kabat et al., Sequences of Proteins of Immunological Interest, 5th Ed. Public Health Service, National Institutes of Health, Bethesda, Md., 1991), Chothia (Chothia et al. (1987) J Mol Biol 196:901-17), IMGT (Lefranc et al. (2003) Dev Comp Immunol 27:55-77) and AbM (Martin and Thornton J Bmol Biol 263:800-15, 1996). The correspondence between the various delineations and variable region numbering is described (see e.g., Lefranc et al. (2003) Dev Comp Immunol 27:55-77; Honegger and Pluckthun, J Mol Biol (2001) 309:657-70; International ImMunoGeneTics (IMGT) database). Available programs such as abYsis by UCL Business PLC can be used to delineate CDRs. The term “CDR”, “HCDR1”, “HCDR2”, “HCDR3”, “LCDR”, “LCDR2” and “LCDR3” as used herein includes CDRs defined by any of the methods described supra, Kabat, Chothia, IMGT or AbM, unless otherwise explicitly stated in the specification.

“Epitope” refers to a portion of an antigen to which an antibody specifically binds. Epitopes typically consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontinuous amino acids that form a conformational spatial unit. For a discontinuous epitope, amino acids from differing portions of the linear sequence of the antigen come in close proximity in 3-dimensional space through the folding of the protein molecule. Antibody “epitope” depends on the methodology used to identify the epitope.

The term “variant” when used in relation to an antigen or an antibody can refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a hK2 variant can result from one or more (such as, for example, about 1 to about 25, about 1 to about 20, about 1 to about 15, about 1 to about 10, or about 1 to about 5) changes to an amino acid sequence of a native hK2. Also by way of example, a variant of an anti-hK2 antibody, such as h11B6, can result from one or more (such as, for example, about 1 to about 25, about 1 to about 20, about 1 to about 15, about 1 to about 10, or about 1 to about 5) changes to an amino acid sequence of a native or previously unmodified anti-hK2 antibody. Variants can be naturally occurring, such as allelic or splice variants, or can be artificially constructed. Polypeptide variants can be prepared from the corresponding nucleic acid molecules encoding the variants. In specific embodiments, the hK2 variant or anti-hK2 antibody variant at least retains hK2 or anti-hK2 antibody functional activity, respectively. In specific embodiments, an anti-hK2 antibody variant binds hK2 and/or is antagonistic to hK2 activity. In certain embodiments, the variant is encoded by a single nucleotide polymorphism (SNP) variant of a nucleic acid molecule that encodes hK2 or anti-hK2 antibody VH or VL regions or subregions, such as one or more CDRs.

A non-limiting example of a variant when used in relation to an antibody is an “Fc variant” which is an antibody having a variant Fc region. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “identity” refers to a relationship between the sequences of two or more polypeptide molecules or two or more nucleic acid molecules, as determined by aligning and comparing the sequences. “Percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

“Specifically binds,” “specific binding,” “specifically binding” or “binds” refer to a protein molecule binding to an antigen or an epitope within the antigen with greater affinity than for other antigens. Typically, the protein molecule binds to the antigen or the epitope within the antigen with an equilibrium dissociation constant (KD) of about 1×10-7 M or less, for example about 5×10-8 M or less, about 1×10-8 M or less, about 1×10-9 M or less, about 1×10-10 M or less, about 1×10-11 M or less, or about 1×10-12 M or less, typically with the KD that is at least one hundred fold less than its KD for binding to a non-specific antigen (e.g., BSA, casein). As used herein, an antibody or antigen binding domain “with binding specificity for hK2” refers to an antibody or antigen binding domain that specifically binds to hK2, respectively.

As used herein, in certain embodiments, the term “subject” refers to a mammal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder. The term “patient” as used herein refers to a human.

“Administer” or “administration” refers to the act of physically delivering a substance as it exists outside the body into a patient, by injection or otherwise, such as by mucosal, intradermal, intravenous, intramuscular, subcutaneous delivery, and/or any other method of physical delivery described herein or known in the art.

As used herein, the terms “treat,” “treatment” and “treating” refer to the reduction or amelioration of the progression, severity, and/or duration of a disease or condition resulting from the administration of one or more therapies. Treating can be determined by assessing whether there has been a decrease, alleviation and/or mitigation of one or more symptoms associated with the underlying disorder such that an improvement is observed with the patient, despite that the patient can still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The term “effective amount” or “therapeutically effective amount” as used herein refers to the amount of a radioconjugate or a pharmaceutical composition provided herein which is sufficient to result in the desired therapeutic effect for a given condition and administration regimen.

The terms medicament, pharmaceutical, active agent, active pharmaceutical ingredient (API), drug, medication, and active are used herein interchangeably to refer to the pharmaceutically active compound(s) in a pharmaceutical composition. An example of an API suitable for use in accordance with the present invention is a radioconjugate with binding specificity for hK2. A pharmaceutical composition can include one or more API(s) and one or more additional ingredients referred to herein as “excipients.” Preferably, the excipients are substantially or completely pharmaceutically inert.

The term “dose” refers to the total amount of a particular pharmaceutical composition administered to a patient at a particular time. Preferably, a dose is delivered as a single administration of a unit dose of pharmaceutical composition (e.g., via an intravenous administration). Alternatively, there can be multiple administrations of a unit dose that has been subdivided into multiple sub-doses (wherein a sub-dose refers to a portion of the unit dose).

The term “pharmaceutically acceptable,” as used herein, means non-toxic and preferably permitted by a regulatory agency, e.g., of a European or U.S. Federal or state government, or listed in the U.S. Pharmacopeia or other generally recognized pharmacopeia for use in animals, and more particularly in humans.

Radioactive decay refers to the process by which an unstable atomic nucleus loses energy by radiation to produce at least one daughter nuclide. Half-life refers to the time required for one half of the atomic nuclei of a radioactive sample to decay to its daughter nuclide. The non-SI unit of measure for radioactivity of a substance is curie (Ci). One curie is equal to that quantity of radioactive material in which the number of atoms decaying per second is equal to 37 billion (3.7×1010). An alternative unit of measure for the radioactivity of a substance is the SI unit of the becquerel (Bq). The becquerel is equal to that quantity of radioactive material in which one atomic nucleus decays per second. Specific activity refers to the amount of radioactivity per unit mols or mass in a sample; for example, sometimes expressed as Ci/mmol or Ci/mg. Radioactive concentration, also known as specific concentration (e.g., expressed as mCi/mL or Ci/mL) refers to the total amount of radioactivity per unit volume.

In reference to immunoconjugates and radioconjugates, the term “conjugated” means “joined.” Molecules (such as an antibody and a chelator) can be joined to each other, for example, by covalent bonding.

“Cancer” refers to a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. A “cancer” or “cancer tissue” can include a tumor.

As used herein, the term “adverse event” (AE) refers to any untoward medical occurrence in a patient administered a pharmaceutical product and which does not necessarily have a causal relationship with the treatment. According to embodiments of the invention, AEs are rated on a 3-point scale of increasing severity using the following definitions: mild (grade 1), referring to an AE that is easily tolerated by the subject, which causes minimal discomfort and does not interfere with everyday activities; moderate (grade 2), referring to an AE that is sufficiently discomforting to interfere with normal everyday activities and intervention can be needed; severe (grade 3), referring to an AE that prevents normal everyday activities, and treatment or other intervention is usually needed. Adverse events include cytokine release syndrome (CRS), immune effector cell associated neurotoxicity syndrome (ICANS), systemic administration related reactions (sARRs), immune-related adverse events (irAEs).

A serious AE (SAE) can be any AE occurring at any dose that results in any of the following outcomes: death, where death is an outcome, not an event; life-threatening, referring to an event in which the patient is at risk of death at the time of the event; it does not refer to an event which could hypothetically have caused death had it been more severe; in patient hospitalization, i.e., an unplanned, overnight hospitalization, or prolongation of an existing hospitalization; persistent or significant incapacity or substantial disruption of the ability to conduct normal life functions; congenital anomaly/birth defect; important medical event (as deemed by the investigator) that can jeopardize the patients or can require medical or surgical intervention to prevent one of the other outcomes listed above (e.g., intensive treatment in an emergency room or at home for allergic bronchospasm or blood dyscrasias or convulsions that do not result in hospitalization). Hospitalization is official admission to a hospital. Hospitalization or prolongation of a hospitalization constitutes criteria for an AE to be serious; however, it is not in itself considered an SAE. In the absence of an AE, hospitalization or prolongation of hospitalization should not be reported as a SAE by the participating investigator. This can be the case, in the following situations: the hospitalization or prolongation of hospitalization is needed for a procedure required by the protocol; or the hospitalization or prolongation of hospitalization is a part of a routine procedure followed by the center (e.g., stent removal after surgery). This should be recorded in the study file. Hospitalization for elective treatment of a pre-existing condition that did not worsen during the study is not considered an AE.

Complications that occur during hospitalization are AEs. If a complication prolongs hospitalization, or meets any of the other SAE criteria, then the event is an SAE.

Embodiments of the present invention relate to compositions and methods for targeting hK2 with a radioconjugate to achieve efficacious cancer cell death (e.g., tumor cell death) in prostate cancer patients. As used herein, an “immunoconjugate” refers to an antibody, or an antigen binding domain, that is conjugated (joined, e.g., bound via a covalent bond) to a second molecule, such as a toxin, drug, radiometal ion, chelator, radiometal complex, etc. A “radioconjugate” (also referred to herein as a “radioimmunoconjugate”) in particular refers to an antibody, or an antigen binding domain, that is conjugated (joined, e.g., bound via a covalent bond) to at least one radiometal complex. Stated another way, a radioconjugate refers to at least one radiometal complex joined, e.g., bound via a covalent bond, to an antibody or antigen binding domain. A radioconjugate can comprise at least one radiometal complex that comprises a linker, wherein the radiometal complex is joined to the antibody or antigen binding domain via the linker.

As used herein, an “antibody-chelator complex” or “conjugate intermediate” or “drug substance intermediate” refers to a precursor of a radioconjugate, which comprises an antibody, or antigen binding domain, that is conjugated (joined, e.g., bound via a covalent bond) to a chelator that does not comprise a radiometal. A conjugate intermediate can comprise a linker, wherein the chelator is joined to the antibody or antigen binding domain via the linker. After a radiometal is chelated to the chelator of a conjugate intermediate, it becomes a radioconjugate. For example, “DOTA-mAb” refers to a conjugate intermediate comprising an DOTA conjugated to an antibody. An example of a conjugate intermediate is DOTA-h11B6. As used herein, “DOTA-h11B6” is a conjugate intermediate which comprises DOTA conjugated to the antibody h11B6, optionally via a linker.

A chelator can be conjugated to an antibody according to methods known in the art; for example, a chelator can be conjugated to an antibody via a linker. Thus, radioconjugates and conjugate intermediates of the present invention can comprise a chelator joined to an antibody by a linker. As used herein, the term linker generally refers to a chemical moiety that joins a chelator to an antibody or antigen binding domain. Any suitable linker known to those skilled in the art in view of the present disclosure can be used in the invention. The linkers can contain, for example, a substituted or unsubstituted alkyl, a substituted or unsubstituted heteroalkyl moiety, a substituted or unsubstituted aryl or heteroaryl, a polyethylene glycol (PEG) linker, a peptide linker, a sugar-based linker, or a cleavable linker, such as a disulfide linkage or a protease cleavage site such as valine-citrulline-p-aminobenzyl (PAB).

A “radiometal complex” as used herein refers to a complex comprising a radiometal ion associated with a chelator that is a macrocyclic compound. Typically, a radiometal ion is bound to or coordinated to a macrocyclic compound via coordinate bonding. Heteroatoms of the macrocyclic ring can participate in coordinate bonding of a radiometal ion to a macrocycle compound. A macrocycle compound can be substituted with one or more substituent groups, and the one or more substituent groups can also participate in coordinate bonding of a radiometal ion to a macrocycle compound in addition to, or alternatively to the heteroatoms of the macrocyclic ring. Other examples of possible linkages between the chelator and radioisotope include guest-hosting binding such as ionic bonding, hydrogen bonding, van der Waals forces or hydrophobic interactions. A radiometal complex can optionally comprise a linker, which is a chemical moiety that joins the chelator to the antibody or antigen binding domain.

As used herein, the terms “radiometal,” “radioisotope,” “radiometal ion” and “radioactive metal ion” are used interchangeably and refer to one or more isotopes of the elements that emit particles and/or photons. Non-limiting examples of radioisotopes that can be used for therapeutic applications include, e.g., beta or alpha emitters, such as, e.g., ²²⁵Ac, ¹⁷⁷Lu, ³²P, ⁴⁷Sc, ⁶⁷Cu, ⁷⁷As, ⁸⁹Sr, ⁹⁰Y, ⁹⁹Tc, ¹⁰⁵Rh, ¹⁰⁹Pd, ¹¹¹Ag, ¹³¹I, ¹³⁴Ce, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²⁵⁵Fm and ²²⁷Th. Other non-limiting examples of radioisotopes that can be used as imaging agents include gamma-emitting radioisotopes, such as, e.g., ¹⁷⁷Lu, ⁶²Cu, ⁶⁴Cu, ⁶⁷Ga, ⁶⁸Ga, ⁸⁶Y, ⁸⁹Zr, and ¹¹¹In. In certain embodiments, the radiometal ion is a “therapeutic emitter,” meaning a radiometal ion that is useful in therapeutic applications. Examples of therapeutic emitters include, but are not limited to, beta or alpha emitters, such as, ¹³²La, ¹³⁵La, ¹³⁴Ce, ¹⁴⁴Nd, ¹⁴⁹Tb, ¹⁵²Tb, ¹⁵⁵Tb, ¹⁵³Sm, ¹⁵⁹Gd, ¹⁶⁵Dy, ¹⁶⁶Ho, ¹⁶⁹Er, ¹⁷⁷Lu, ¹⁸⁶Re, ¹⁸⁸Re, ¹⁹⁴Ir, ¹⁹⁸Au, ¹⁹⁹Au, ²¹¹At, ²¹²Pb, ²¹²Bi, ²¹³Bi, ²²³Ra, ²²⁵Ac, ²⁵⁵Fm and ²²⁷Th, ²²⁶Th, ²³⁰U. Preferably, a radiometal ion used in the invention is an alpha-emitting radiometal ion, such as actinium-225 (²²⁵Ac).

It is noted that certain radiometals can be used as therapeutic agents (e.g., ²²⁵Ac) and/or as imaging agents (e.g., ¹¹¹In). A suitable radiometal for use as a therapeutic agent is one that is capable of reducing or inhibiting the growth of, or in particular killing, a cancer cell, such as a prostate cancer cell. In certain embodiments, radioconjugates of the present invention can deliver a cytotoxic payload with the ability to emit alpha and/or beta particles in the vicinity of a tumor by binding onto cancer cells' surface antigens and initiating cell death. In certain embodiments, the radioconjugates of the present invention are internalized into hk2-expressing cancer cells.

The term “225Ac,” “225 Ac,” or “Ac-225” as used herein refer to actinium-225 which is an alpha-emitting radiometal. According to particular embodiments, the approximate ten-day half-life of ²²⁵Ac (about 9.9 days) is long enough to be able to prepare the compounds described herein, but short enough to match the circulation pharmacokinetics of the antibody that is conjugated to the radiometal complex, such as h11B6. ²²⁵Ac decays in a series of steps that ultimately emits four alpha particles before reaching a stable isotope, ²⁰⁹Bi, thereby providing an increased potency of the compounds.

The radioconjugate of the present invention comprises an antibody that is an h11B6 antibody. Embodiments of an h11B6 antibody are described in WO 2015/075445, U.S. Pat. Nos. 10,100,125, and 11,230,609, which are incorporated by reference herein. As used herein, an “h11B6 antibody” or “h11B6 mAb” or “h11B6” or “hu11B6” refers to an antibody with binding specificity for human kallikrein-2 (hK2). The radioconjugate of the present invention comprises an h11B6 antibody that is an IgG1 antibody. The radioconjugate of the present invention comprises an h11B6 antibody that is an IgG1 kappa isotype. The radioconjugate of the present invention comprises an h11B6 antibody that is an IgG1 antibody or a variant thereof, such as an Fc variant. The radioconjugate of the present invention comprises an antibody conjugated to DOTA, optionally via a linker. For example, the h11B6 antibody can be conjugated to DOTA to produce a DOTA-h11B6 conjugate intermediate, and DOTA-h11B6 is then chelated to ²²⁵Ac to produce the radioconjugate ²²⁵Ac-DOTA-h11B6. DOTA-h11B6 is formed by chemically conjugating h11B6 to a DOTA, optionally via a linker. For example, DOTA-h11B6 can be formed by chemically conjugating h11B6 to a DOTA derivative, p-SCN-Bn-DOTA (CAS Registry Number: 127985-74-4; Chemical Name: 2-S-(4-Isothiocyanatobenzyl)-1,4,7,10-tetraazacyclododecane-1,4,7,10-tetraacetic acid) according to known methods. One or multiple DOTA molecules can be chemically linked to h11B6, such as to the Fc region of h11B6. For example, a DOTA can be chemically linked to the epsilon amino group of lysine side chains of the h11B6 mAb. DOTA-h11B6 can then be chelated to ²²⁵Ac to produce the radioconjugate ²²⁵Ac-DOTA-h11B6. When administered to a patient, the radioconjugate ²²⁵Ac-DOTA-h11B6 can bind and internalize within hK2-expressing cells.

As used herein, “time of dosing” refers to the time at which a patient is administered a dose of the pharmaceutical composition comprising the radioconjugate (e.g., whether as a single administration or in multiple administrations of more than one sub-dose). Due to the decay of ²²⁵Ac, the amount of radioactivity provided by ²²⁵Ac in the pharmaceutical composition decreases from the time of manufacture to the time of dosing, i.e., from the approximate time that ²²⁵Ac is chelated to the conjugate intermediate to form the radioconjugate during the manufacturing process to the time that the radioconjugate is administered to a patient. According to an example, if the radioactivity provided by ²²⁵Ac in the pharmaceutical composition is about 264 μCi at the approximate time that ²²⁵Ac is chelated to the conjugate intermediate to form the radioconjugate (e.g., after the radioconjugate is formed and purified), the radioactivity about 96 hours later at the time of dosing can be about 200 μCi. The decay and therefore the amount of ²²⁵Ac at any given time can be calculated based on the initial amount of activity measured at time zero, the amount of time elapsed and the half-life of ²²⁵Ac.

As used herein, a “targeted” specific activity or “targeted” radioactivity or “targeted” radioactive concentration of the radiometal refers to the amount of specific activity or radioactivity or radioactive concentration, respectively, that is calculated to be present in a dose of pharmaceutical composition at the anticipated time of administration to the patient, e.g., based on the amount of radiometal present in the composition at the time of manufacture and the amount of time (and accompanying radiometal decay) expected between manufacture and administration to the patient. It should be appreciated that the actual specific activity or radioactivity or radioactive concentration at the time of dosing can vary slightly from the targeted specific activity or radioactivity or radioactive concentration, respectively (for example, in the event that the actual time of administration to a patient differs slightly from the anticipated time of administration).

“Androgen receptor pathway inhibitor” (ARPI) refers to a substance that keeps androgens from binding to proteins called androgen receptors, which are found in normal prostate cells, some prostate cancer cells, and in cells of some other tissues. Preventing this binding blocks the effects of these hormones in the body. Treatment with androgen receptor blockers can keep prostate cancer cells from growing. Examples of androgen receptor blockers used to treat prostate cancer are apalutamide, bicalutamide, darolutamide, enzalutamide, flutamide, and nilutamide. Also called androgen receptor antagonist and antiandrogen.

As used herein “nadir” is defined as the lowest PSA level measured after the prior dose, including the PSA measured immediately prior to that dose. The PSA level can be measured from the blood or serum of the patient using methods known in the art in view of the present disclosure.

As used herein, “PSA progression” refers to the PSA level measured at a point during a treatment that has ≥25% and/or ≥2 ng/ml above a reference PSA level. Examples of the reference blood PSA level can include, but are not limited to, the blood PSA level measured immediately before the initiation of the treatment, the blood PSA level measured immediately after the treatment, the blood PSA level measured prior to any administering of the radioconjugate (pretreatment baseline level), or the nadir.

Pharmaceutical compositions of the present invention can be administered via any suitable route known to those skilled in the art. For example, the compositions can be administered parenterally. Non-limiting examples of routes of administration include intravenous (IV), intramuscular or subcutaneous, or they can be administered by infusion techniques. In certain aspects, the methods of treatment herein comprise injecting the pharmaceutical composition intravenously.

A dose of a pharmaceutical composition of the present invention can be administered to the patient by a single administration, or by administering the dose in multiple administrations of more than one sub-dose (e.g., by administering the dose in multiple subdivisions of the dose). Alternatively, the dose can be provided as a continuous infusion over a prolonged period.

It will be appreciated by persons skilled in the art that pharmaceutical compositions of the present invention can be administered alone or in combination with one or more additional therapeutic agents or imaging agents or modalities as determined by the attending physician. A pharmaceutical composition of the present invention can be administered to the patient before or concurrently with other therapeutic modalities for the treatment of prostate cancer.

Preferably, pharmaceutical compositions of the present invention are in the form of a sterile aqueous solution which can contain other substances to make the solution isotonic with blood and/or to provide a suitable pH. In some embodiments, the pH of the aqueous solution is about 4 to about 7, about 4.5 to about 6.5, about 5 to about 6, or about 5.5. In other embodiments, the pH of the aqueous solution is about 5, about 5.1, about 5.2, about 5.3, about 5.4, about 5.5, about 5.6, about 5.7, about 5.8, about 5.9, or about 6. In further embodiments, the pH of the aqueous solution is about 5.5.

As noted herein, due to the decay of ²²⁵Ac, the amount of radioactivity provided by ²²⁵Ac in a dose of the pharmaceutical composition decreases from the time of manufacture (from the approximate time that ²²⁵Ac is chelated to the conjugate intermediate to form the radioconjugate) to the time that the dose is administered to a patient. Preferably, the radioconjugate is labeled with a sufficient amount of ²²⁵Ac during manufacture to account for the decrease in specific activity of ²²⁵Ac that is estimated to occur between the approximate time of chelation and the time of dosing a patient. It is also preferable to limit the amount of time between formation of the radioconjugate (via chelation of ²²⁵Ac) to administration of the dose to the patient.

The excipients can be selected by one skilled in the art and can take a wide variety of forms depending upon the desired route of administration. For example, for parenteral administration, the excipients can include sterile water, and other ingredients can be added to increase solubility and preservation of the composition. Injectable suspensions or solutions can also be prepared utilizing excipients that comprise aqueous carriers and/or appropriate additives such as, solubilizers and preservatives.

The pharmaceutical compositions can be subjected to conventional pharmaceutical operations such as sterilization and/or can contain conventional adjuvants. Pharmaceutical compositions can also contain aqueous and non-aqueous sterile injection solutions which can contain antioxidants, buffers, bacteriostats and/or solutes which render the formulation isotonic with the blood of the intended recipient.

In a general aspect the present application provides a method of treating prostate cancer, preferably advanced prostate cancer, preferably metastatic castration-resistant prostate cancer (mCRPC), in a patient in need thereof, comprising:
i) administering to the patient a (therapeutically effective) first dose of a radioconjugate comprising an antibody or an antigen binding fragment having binding specificity for hK2 conjugated to a chelator, optionally via a linker, wherein actinium-225 (225Ac) is chelated to the chelator; wherein no prostate-specific antigen (PSA) progression relative to a serum PSA level of the patient measured prior to the administering of (i), preferably relative prior to any administering of the radioconjugate (pretreatment baseline level), is observed for a period of time after the administering of the first dose, preferably for a period of at least 12 weeks after the administering of the first dose; and
ii) administering to the patient a (therapeutically effective) second dose of the radioconjugate after the period of time if (i), if the following conditions are met (before the administering of the second dose):
   a. there is a PSA progression after the period of time of (i) relative to the lowest serum PSA level of the patient (nadir) from immediately prior to the administering of (i) to the end of the period of time; and
   b. the patient has a platelet count above 100×10E9 per liter of blood;
iii) optionally repeating (ii).

In some embodiments, during the period of time of (i), there is no administering of another dose of the radioconjugate (other than the first dose of (i)).

In some embodiments, the period of time in (i) is 12 weeks or more than 12 weeks, such as 12-96 weeks, or 12-80 weeks, or 12-60 weeks, or 12-48 weeks, or 12-40 weeks, for example 12 or 38 weeks.

In some embodiments, the method further comprising monitoring the serum PSA level of the patient after the administering of step (i), such as measuring or obtaining a measurement of the serum PSA level of the patient one or more times after the administering of step (i), for example, the serum PSA level is measured once every 4-6 weeks, preferably once every 4 weeks, such as three times every 4 weeks, and optionally further comprising measuring or obtaining the measurement of the serum PSA level of the patient immediately before the administering of (i).

In some embodiments, the second dose of the radioconjugate of (ii) is not administered to the patient, if there is no PSA progression relative to the lowest serum PSA level (nadir) of the patient, and/or if the patient has a platelet count of 100×10E9 per liter of blood or lower.

In some embodiments, there has been no PSA progression in (i), when any serum PSA level measured during the period is not 25% or more higher than the serum PSA level of the patient measured prior to (i), preferably prior to any administering of the radioconjugate (pretreatment baseline level).

In some embodiments, there has been no PSA progression in (i), when any serum PSA level measured during the period is not 2 ng/ml or more higher than the serum PSA level of the patient measured prior to (i), preferably prior to any administering of the radioconjugate (pretreatment baseline level).

In some embodiments, there has been no PSA progression in (i), when any serum PSA level measured during the period is not 25% or more and not 2 ng/mL or more higher than the serum PSA level of the patient measured prior to step (i), preferably prior to any administering of the radioconjugate (pretreatment baseline level).

In some embodiments, there is a PSA progression in (ii)a, when a serum PSA level measured after the period is 25% or more higher than the lowest serum PSA level of the patient measured over the period of (i) or immediately prior to the administering of step (i).

In some embodiments, there is a PSA progression in (ii)a, when a serum PSA level measured after the period is 2 ng/mL or more higher than the lowest serum PSA level of the patient measured over the period of (i) or immediately prior to the administering of step (i).

In some embodiments, there is a PSA progression in (ii)a, when a serum PSA level measured after the period is 25% or more and 2 ng/ml or more higher than the lowest serum PSA level of the patient measured over the period of (i) or immediately prior to the administering of step (i).

In some embodiments, the dose of the radioconjugate administered in step (i) is 100-400 μCi, such as a dose of 150 μCi, 200 μCi, 250 μCi, 300 μCi or 400 μCi, preferably 150-300 μCi, preferably 200-250 μCi, such as 200 μCi or 250 μCi, preferably 250 μCi.

In some embodiments, the first dose of the radioconjugate administered in (i) is 150 μCi, 200 μCi or 250 μCi.

In some embodiments, the first dose of the radioconjugate administered in (i) is 200 μCi or 250 μCi, preferably 250 μCi.

In some embodiments, the first dose of the radioconjugate administered in (i) is 150 μCi or 200 μCi, preferably 150 μCi.

In some embodiments, the patient has received one or more doses of the radioconjugate prior to step (i).

In some embodiments, the patient has not received any dose of the radioconjugate prior to step (i).

In some embodiments, the second dose of the radioconjugate administered in step (ii) is 100-250 μCi, such as 100 μCi, 150 μCi, 200 μCi, or 250 μCi, preferably 100 or 150 μCi.

In some embodiments, the second dose of the radioconjugate administered in step (ii) is 100 or 150 μCi.

In some embodiments, the patient has not received any dose of the radioconjugate prior to step (i), wherein the first dose of the radioconjugate administered in step (i) is 200 μCi or 250 μCi, preferably 250 μCi, and wherein the second dose of the radioconjugate administered in step (ii) is 100 or 150 μCi.

In some embodiments, the patient has not received any dose of the radioconjugate prior to step (i), wherein the first dose of the radioconjugate administered in step (i) is 150 μCi or 200 μCi, preferably 150 μCi, and wherein the second dose of the radioconjugate administered in step (ii) is 100 or 150 μCi.

In some embodiments, the second dose of the radioconjugate administered in step (ii) is 150 μCi, if cumulative exposure to the radioconjugate for the patient prior to step (ii) is preferably not higher than 500 μCi, or is not higher than 450 μCi.

In some embodiments, the second dose of the radioconjugate administered in step (ii) is 100 μCi, if cumulative exposure to the radioconjugate for the patient prior to step (ii) is 500 μCi.

In certain embodiments, the second dose of the radioconjugate administered in step (ii) is lower than the dose of the radioconjugate administered in step (i).

In some embodiments, the method further comprises (iii) administering to the patient a third dose of the radioconjugate after a (second) period of time after (ii), preferably at least 12 weeks after (ii), if the following conditions are met: a) there is a PSA progression after the (second) period, relative to the lowest serum PSA level of the patient (nadir) measured over the (second) period or immediately prior to the administering of (ii); and b) the patient has a platelet count above 100×10E9 per liter of blood.

In some embodiments, the cumulative exposure to the radioconjugate does not exceed 600 μCi, preferably does not exceed 500 μCi.

In some embodiments, the antibody or an antigen binding fragment comprises a heavy chain complementarity determining region (HCDR) 1, a HCDR2 and a HCDR3 of a heavy chain variable region (VH) comprising the amino acid sequence of SEQ ID NO: 7, and a light chain complementarity determining region (LCDR) 1, a LCDR2, and a LCDR3 of a light chain variable region (VL) comprising the amino acid sequence of SEQ ID NO: 8, wherein the HCDR1, HCDR2, HCDR3, LCDR1, LCDR2 and LCDR3 are defined by the Kabat, Chothia, IMGT or AbM numbering system.

In certain embodiments, the antibody or an antigen binding fragment comprises the HCDR1, HCDR2, HCDR3, LCDR1, LCDR2 and LCDR3 having the amino acid sequences of SEQ ID NOs: 1, 2, 3, 4, 5, and 6, respectively.

In certain embodiments, the antibody or an antigen binding fragment comprises the VH and VL having the amino acid sequences at least 90% identical to SEQ ID NO: 7 and SEQ ID NO: 8, respectively. Preferably, the antibody or an antigen binding fragment comprises the VH and VL having the amino acid sequences of SEQ ID NO: 7 and SEQ ID NO: 8, respectively.

In certain embodiments, the antibody comprises a heavy chain sequence and a light chain sequence having the amino acid sequences at least 90% identical to SEQ ID NO: 11 and SEQ ID NO: 12, respectively. Preferably, the antibody comprises a heavy chain sequence and a light chain sequence having the amino acid sequences of SEQ ID NO: 11 and SEQ ID NO: 12, respectively.

In some embodiments, the chelator is 1,4,7,10-tetraazacyclododecane-1,4,7,10,tetraacetic acid (DOTA).

In some embodiments, the linker comprises or is a benzyl isothiocyanate, such as 4-isothiocyanatobenzyl, 3-isothiocyanatobenzyl, 2-isothiocyanatobenzyl, wherein the benzyl isothiocyanate optionally has one or more alkyl substituents and/or one or more cyclic acid substituents, e.g., wherein the benzyl isothiocyanate optionally has substituents L1 and/or L2 at the benzylic position, wherein:
L1 is H or an alkyl or cyclic alkyl (when L1 is an alkyl or cyclic alkyl, the alkyl or cyclic alkyl can have carbon or heteroatom substitutions);
L2 is H or an alkyl or cyclic alkyl. (when L1 is an alkyl or cyclic alkyl, the alkyl or cyclic alkyl can have carbon or heteroatom substitutions); or
L1 and L2 form a cycle, such as a carbon cycle or a heterocycle or a fused cycle.

In some embodiments, the antibody or antigen binding fragment is conjugated to the chelator via a linker.

In some embodiments, the radioconjugate comprises the antibody or antigen binding fragment conjugated to 2-(4-isothiocyanatobenzyl)-1,4,7,10-tetraazacyclododecane-1,4,7,10-tetraacetic acid with the chelated actinium-225 (²²⁵Ac), preferably, the 2-(4-isothiocyanatobenzyl)-1,4,7,10-tetraazacyclododecane-1,4,7,10-tetraacetic acid is covalently linked to one or more amino acid residues in the Fc region of the antibody.

In some embodiments, the radioconjugate comprises the antibody or antigen binding fragment conjugated to 2-S-(4-isothiocyanatobenzyl)-1,4,7,10-tetraazacyclododecane-1,4,7,10-tetraacetic acid with the chelated actinium-225 (225Ac), preferably, the 2-S-(4-isothiocyanatobenzyl)-1,4,7,10-tetraazacyclododecane-1,4,7,10-tetraacetic acid is covalently linked to one or more amino acid residues in the Fc region of the antibody.

In some embodiments, the radioconjugate comprises an average of from about 1 to about 4 chelator molecules conjugated to the antibody.

In some embodiments, the radioconjugate provides a specific activity from about 50 μCi to about 350 μCi, such as 50 μCi, 100 μCi, 150 μCi, 200 μCi, per about 2-3 mg of total antibody.

In some embodiments, the radioconjugate is administered in a pharmaceutical composition, preferably in a pharmaceutical composition formulated for intravenous administration, wherein the pharmaceutical composition comprises the radioconjugate and one or more pharmaceutically acceptable excipients.

In some embodiments, the pharmaceutical composition comprises the radioconjugate, a radioprotectant, surfactant and buffer, with a pH of 5-6.

In certain embodiments, the pharmaceutical composition further comprises a conjugate comprising the antibody or the antigen binding fragment conjugated to the chelator without actinium-225 (²²⁵Ac) chelated by the chelator.

In certain embodiments, the pharmaceutical composition is administered with a total antibody mass of 2 to 10 mg per dose.

In certain embodiments, the pharmaceutical composition is administered at 12.5 μCi/mL, 25 μCi/mL, 37.5 μCi/mL or 50 μCi/mL (at the time of dosing).

In some embodiments, the radioconjugate or the pharmaceutical composition is administered to the patient intravenously.

In some embodiments, the patient:
1) has a prior treatment with at least one androgen receptor pathway inhibitor (ARPI);
2) can have a prior chemotherapy treatment;
3) has not had prior radioconjugate therapy; and/or
4) has no prior radiotherapy with >30% red marrow distribution.

In some embodiments, the patient has received 0 to 12, for example 4, prior lines of therapy, such as ARPI and/or taxane.

In some embodiments, the patient has met one or more, such as all, of the following criteria:
1) mCRPC with histologic confirmation of adenocarcinoma;
2) prior exposure to at least one androgen receptor-targeted therapy (eg, abiraterone acetate, enzalutamide, apalutamide, darolutamide), or prior taxane or other chemotherapy;
3) treatment with other agents for prostate cancer, if received, discontinued ≥2 weeks prior to first dose of the radioconjugate;
4) prior orchiectomy or medical castration; or, for patients who have not undergone orchiectomy, receiving ongoing androgen deprivation therapy with a gonadotropin releasing hormone (GnRH) analog (agonist or antagonist) prior to the first dose of the radioconjugate;
5) palliative radiotherapy (eg, soft tissue lesions) completed >2 weeks prior to the first dose of the radioconjugate, except for palliative radiotherapy for pain (eg, bone pain), which may be used any time prior to first dose;
6) Eastern Cooperative Oncology Group (ECOG) performance status of 0 or 1;
7) Hematology laboratory parameters within the following range:
a. Hemoglobin >9.0 g/dL
   b. Absolute neutrophil count >1.5×10⁹/L
   c. Platelet count >100×10⁹/L
8) Clinical chemistry laboratory parameters within the following range before the first dose of study drug:
   a. Serum total bilirubin <1.5×upper limit of normal (ULN)
   b. Aspartate aminotransferase (AST) and alanine aminotransferase (ALT)≤2.5×ULN (or ≤4×ULN for participants with tumor involvement in the liver)
   c. Calculated or measured creatinine clearance >50 mL/min/1.73 m² as determined by Modification of Diet in Renal Disease formula
   d. Grade ≤1 proteinuria. For proteinuria clearly attributable to other known medical conditions of the participant, such as hemorrhagic radiation cystitis or chronic hematuria caused by tumor infiltrates, Grade ≥1 proteinuria requires an approval from the sponsor's medical monitor.

In some embodiments, the patient does not have one or more of the following:
1) Prior treatment with radium Xofigo® (Ra 223 dichloride), strontium, or samarium therapy or radioconjugate therapy;
2) Prior radiation therapy encompassing >30% of expected red marrow distribution;
3) Diffuse bone or bone-marrow involvement (ie, superscan—diffuse increased marrow uptake with reduced background and renal activity);
4) Spinal cord compression (unless treated, stable, and approved by the sponsor medical monitor);
5) Active CNS metastases;
6) Known history of myelodysplastic syndrome, leukemia, or hematological malignancy with features suggestive of myelodysplastic syndrome/acute myeloid leukemia at any timepoint;
7) Toxicity from prior anticancer therapy has not resolved to baseline levels or to Grade ≤1 (except alopecia, radiation tissue fibrosis, or peripheral neuropathy);
8) Malignancy diagnosis other than the disease under study within 2 years prior to the first dose of study drug, except for squamous and basal cell carcinoma of the skin, non-muscle invasive bladder cancer, or any malignancy considered cured or has minimal risk of recurrence within 1 year of first dose of study drug (in the opinion of both the investigator and sponsor's medical monitor);
9) Any systemic anti-neoplastic therapy (eg, chemotherapy, immunotherapy or biological therapy [including mAbs], poly ADP ribose polymerase [PARP] inhibitors)≤30 days prior to the first dose of the radioconjugate except for luteinizing hormone-releasing hormone agonists/antagonists or GnRH agonists/antagonists, and androgen axis drugs (e.g., enzalutamide, apalutamide, darolutamide, and/or abiraterone acetate plus prednisone) must have been discontinued ≥2 weeks prior to the first dose of the radioconjugate.

In some embodiments, the treatment provides a lower rate of severe toxicities (toxicity of Grade 3 or higher according to the NCI CTCAE version 5.0), e.g., compared to the administering of the first dose of (i) at a fixed dose level and at a fixed dose schedule (e.g., compared to the administering of the first dose of (i) once every 8 weeks).

In some embodiments, the treatment results in effective treatment of the prostate cancer, as measured by the PSA response, and/or Overall response rate (ORR) according to response criteria of Prostate Cancer Working Group 3 (PCWG3).

In some embodiments, the prostate cancer is locally advanced prostate cancer, metastatic castration-sensitive prostate cancer (CSPC), metastatic castration-resistant prostate cancer (CRPC), non-metastatic CSPC, non-metastatic CRPC, or metastatic prostate cancer, preferably the prostate cancer is metastatic CRPC.

In some embodiments, the prostate cancer is metastatic hormone sensitive prostate cancer (mHSPC). In some embodiments, the prostate cancer is metastatic hormone sensitive prostate cancer with non-castrate levels of testosterone, for example with levels of testosterone >150 ng/dL. In some embodiments, the patient does not have undergone orchiectomy and is not receiving ongoing androgen deprivation therapy with a gonadotropin releasing hormone (GnRH) analog (agonist or antagonist) prior to the first dose of the radioconjugate. In some embodiments, the mHSPC is a mHSPC with no (evidence of) metastasis to visceral organs. In some embodiments, the mHSPC is a mHSPC with metastasis in no more than 5 (distinct) locations in the patient's body. In some embodiments, the mHSPC is a mHSPC, which can be treated or which can be encompassed by no more than 5 radiation isocenters (i.e., no more than five radiation fields). In some embodiments, the mHSPC is a mHSPC with metastasis in no more than 5 (distinct) locations in the patient's body, and the mHSPC is a mHSPC, which can be treated or which can be encompassed by no more than 5 radiation isocenters (i.e., no more than five radiation fields). In some embodiments, the mHSPC is a mHSPC with no more than five (distinct) metastases (e.g., no more than five lesions on PSMA-PET scan). In some embodiments, the mHSPC is a mHSPC with metastasis in no more than 5 (distinct) locations in the patient's body and with no (evidence of) metastasis in visceral organs.

In some embodiments, the prostate cancer is metastatic hormone sensitive prostate cancer (mHSPC), more particularly metastatic hormone sensitive prostate cancer with non-castrate levels of testosterone, for example with levels of testosterone >150 ng/dL; and the patient does not have undergone orchiectomy and is not receiving ongoing androgen deprivation therapy with a gonadotropin releasing hormone (GnRH) analog (agonist or antagonist) prior to the first dose of the radioconjugate.

In some embodiments, the prostate cancer is metastatic hormone sensitive prostate cancer (mHSPC), more particularly metastatic hormone sensitive prostate cancer with non-castrate levels of testosterone, for example with levels of testosterone >150 ng/dL; the patient does not have undergone orchiectomy and is not receiving ongoing androgen deprivation therapy with a gonadotropin releasing hormone (GnRH) analog (agonist or antagonist) prior to the first dose of the radioconjugate; and the mHSPC is a mHSPC with no (evidence of) metastasis to visceral organs.

In some embodiments, the prostate cancer is metastatic hormone sensitive prostate cancer (mHSPC), more particularly metastatic hormone sensitive prostate cancer with non-castrate levels of testosterone, for example with levels of testosterone >150 ng/dL; the patient does not have undergone orchiectomy and is not receiving ongoing androgen deprivation therapy with a gonadotropin releasing hormone (GnRH) analog (agonist or antagonist) prior to the first dose of the radioconjugate; and the mHSPC is a mHSPC with metastasis in no more than five (distinct) locations in the patient's body.

In some embodiments, the prostate cancer is metastatic hormone sensitive prostate cancer (mHSPC), more particularly metastatic hormone sensitive prostate cancer with non-castrate levels of testosterone, for example with levels of testosterone >150 ng/dL; the patient does not have undergone orchiectomy and is not receiving ongoing androgen deprivation therapy with a gonadotropin releasing hormone (GnRH) analog (agonist or antagonist) prior to the first dose of the radioconjugate; the mHSPC is a mHSPC with metastasis in no more than five (distinct) locations in the patient's body and with no (evidence of) metastasis to visceral organs.

In another general aspect the present application provides the radioconjugate as defined in any one of the foregoing embodiments for use in the method of any one of the preceding embodiments.

In another general aspect the present application provides the pharmaceutical composition as defined in certain previous embodiments.

In a final general aspect the present application provides, a product comprising the radioconjugate as defined in any one of the preceding embodiments for use in the method of any one of the preceding embodiments.

### SUBJECT MATTER DESCRIBED IN THE APPLICATION

The subject matter described in the application includes, for example:

1. A method of treating prostate cancer, preferably advanced prostate cancer, preferably metastatic castration-resistant prostate cancer (mCRPC), in a patient in need thereof, comprising:
(i) administering to the patient a (therapeutically effective) first dose of a radioconjugate comprising an antibody or an antigen binding fragment having binding specificity for hK2 conjugated to a chelator, optionally via a linker, wherein actinium-225 (225 Ac) is chelated to the chelator; wherein no prostate-specific antigen (PSA) progression relative to a serum PSA level of the patient measured prior to the administering of (i), preferably relative prior to any administering of the radioconjugate (pretreatment baseline level), is observed for a period of time after the administering of the first dose, preferably for a period of at least 12 weeks after the administering of the first dose; and
(ii) administering to the patient a (therapeutically effective) second dose of the radioconjugate after the period of time if (i), if the following conditions are met (before the administering of the second dose):
   a. there is a PSA progression after the period of time of (i), relative to the lowest serum PSA level of the patient (nadir) from immediately prior to the administering of (i) to the end of the period of time; and
   b. the patient has a platelet count above 100×10E9 per liter of blood;
(iii) optionally repeating (ii).
   2. The method of embodiment 1, wherein during the period of time of (i), there is no administering of another dose of the radioconjugate (other than the first dose of (i)).
   3. The method of embodiment 1 or 2, wherein the period of time in (i) is 12 weeks or more than 12 weeks, such as 12-96 weeks, or 12-80 weeks, or 12-60 weeks, or 12-48 weeks, or 12-40 weeks, for example 12 or 38 weeks.
   4. The method of embodiment 1 or 2, further comprising monitoring the serum PSA level of the patient after the administering of step (i), such as measuring or obtaining a measurement of the serum PSA level of the patient one or more times after the administering of step (i), for example, the serum PSA level is measured once every 4-6 weeks, preferably once every 4 weeks, such as three times every 4 weeks, and optionally further comprising measuring or obtaining the measurement of the serum PSA level of the patient immediately before the administering of (i).
   5. The method of any one of the foregoing embodiments, wherein the second dose of the radioconjugate of (ii) is not administered to the patient, if there is no PSA progression relative to the lowest serum PSA level (nadir) of the patient, and/or if the patient has a platelet count of 100×10E9 per liter of blood or lower.
   6. The method of any one of the foregoing embodiments, wherein there has been no PSA progression in (i), when any serum PSA level measured during the period is not 25% or more higher than the serum PSA level of the patient measured prior to (i), preferably prior to any administering of the radioconjugate (pretreatment baseline level).
   7. The method of any of the foregoing embodiments, wherein there has been no PSA progression in (i), when any serum PSA level measured during the period is not 2 ng/ml or more higher than the serum PSA level of the patient measured prior to (i), preferably prior to any administering of the radioconjugate (pretreatment baseline level).
   8. The method of any of the foregoing embodiments, wherein there has been no PSA progression in (i), when any serum PSA level measured during the period is not 25% or more and not 2 ng/ml or more higher than the serum PSA level of the patient measured prior to step (i), preferably prior to any administering of the radioconjugate (pretreatment baseline level).
   9. The method of any of the foregoing embodiments, wherein there is a PSA progression in (ii)a, when a serum PSA level measured after the period is 25% or more higher than the lowest serum PSA level of the patient measured over the period of (i) or immediately prior to the administering of step (i).
   10. The method of any of the foregoing embodiments, wherein there is a PSA progression in (ii)a, when a serum PSA level measured after the period is 2 ng/mL or more higher than the lowest serum PSA level of the patient measured over the period of (i) or immediately prior to the administering of step (i).
   11. The method of any of the foregoing embodiments, wherein there is a PSA progression in (ii)a, when a serum PSA level measured after the period is 25% or more and 2 ng/ml or more higher than the lowest serum PSA level of the patient measured over the period of (i) or immediately prior to the administering of step (i).
   12. The method of any of the foregoing embodiments, wherein the dose of the radioconjugate administered in step (i) is 100-400 μCi, such as a dose of 150 μCi, 200 μCi, 250 μCi, 300 μCi or 400 μCi, preferably 150-300 μCi, preferably 200-250 μCi, such as 200 μCi or 250 μCi, preferably 250 μCi.
   13. The method of any of the foregoing claims, wherein the first dose of the radioconjugate administered in (i) is 150 μCi, 200 μCi or 250 μCi.
   14. The method of any of the foregoing embodiments, wherein the first dose of the radioconjugate administered in (i) is 200 μCi or 250 μCi, preferably 250 μCi.
   15. The method of any of the foregoing claims, wherein the first dose of the radioconjugate administered in (i) is 150 μCi or 200 μCi, preferably 150 μCi.
   16. The method of any of the foregoing embodiments, wherein the patient has received one or more doses of the radioconjugate prior to step (i).
   17. The method of any of the foregoing embodiments, wherein the patient has not received any dose of the radioconjugate prior to step (i).
   18. The method of any of the foregoing embodiments, where the second dose of the radioconjugate administered in step (ii) is 100-250 μCi, such as 100 μCi, 150 μCi, 200 μCi, or 250 μCi, preferably 100 or 150 μCi.
   19. The method of any of the foregoing embodiments, wherein the second dose of the radioconjugate administered in step (ii) is 100 or 150 μCi.
   20. The method of any of the foregoing embodiments, wherein the patient has not received any dose of the radioconjugate prior to step (i), wherein the first dose of the radioconjugate administered in step (i) is 200 μCi or 250 μCi, preferably 250 μCi, and wherein the second dose of the radioconjugate administered in step (ii) is 100 or 150 μCi.
   21. The method of any of the foregoing claims, wherein the patient has not received any dose of the radioconjugate prior to step (i), wherein the first dose of the radioconjugate administered in step (i) is 150 μCi or 200 μCi, preferably 150 μCi, and wherein the second dose of the radioconjugate administered in step (ii) is 100 or 150 μCi.
   22. The method of any of the foregoing embodiments, wherein the second dose of the radioconjugate administered in step (ii) is 150 μCi, if cumulative exposure to the radioconjugate for the patient prior to step (ii) is preferably not higher than 500 μCi, or is not higher than 450 μCi.
   23. The method of any of the foregoing embodiments, wherein the second dose of the radioconjugate administered in step (ii) is 100 μCi, if cumulative exposure to the radioconjugate for the patient prior to step (ii) is 500 μCi.
   24. The method of any of the foregoing embodiments, wherein the second dose of the radioconjugate administered in step (ii) is lower than the dose of the radioconjugate administered in step (i).
   25. The method of any of the foregoing embodiments, further comprising
(iii) administering to the patient a third dose of the radioconjugate after a (second) period of time after (ii), preferably at least 12 weeks after (ii), if the following conditions are met:
   a. there is a PSA progression after the (second) period, relative to the lowest serum PSA level of the patient (nadir) measured over the (second) period or immediately prior to the administering of (ii); and
   b. the patient has a platelet count above 100×10E9 per liter of blood.
      26. The method of any of the foregoing embodiments, wherein cumulative exposure to the radioconjugate does not exceed 600 μCi, preferably does not exceed 500 μCi.
      27. The method of any of the foregoing embodiments, wherein the antibody or an antigen binding fragment comprises a heavy chain complementarity determining region (HCDR) 1, a HCDR2 and a HCDR3 of a heavy chain variable region (VH) comprising the amino acid sequence of SEQ ID NO: 7, and a light chain complementarity determining region (LCDR) 1, a LCDR2, and a LCDR3 of a light chain variable region (VL) comprising the amino acid sequence of SEQ ID NO: 8, wherein the HCDR1, HCDR2, HCDR3, LCDR1, LCDR2 and LCDR3 are defined by the Kabat, Chothia, IMGT or AbM numbering system.
      28. The method of any of the foregoing embodiments, wherein the antibody or an antigen binding fragment comprises the HCDR1, HCDR2, HCDR3, LCDR1, LCDR2 and LCDR3 having the amino acid sequences of SEQ ID NOs: 1, 2, 3, 4, 5, and 6, respectively.
      29. The method of any of the foregoing embodiments, wherein the antibody or an antigen binding fragment comprises the VH and VL having the amino acid sequences at least 90% identical to SEQ ID NO: 7 and SEQ ID NO: 8, respectively.
      30. The method of any of the foregoing embodiments, wherein the antibody comprises a heavy chain sequence and a light chain sequence having the amino acid sequences at least 90% identical to SEQ ID NO: 11 and SEQ ID NO: 12, respectively.
      31. The method of any of the foregoing embodiments, wherein the chelator is 1,4,7,10-tetraazacyclododecane-1,4,7,10,tetraacetic acid (DOTA).
      32. The method of any of the foregoing embodiments, wherein the linker comprises or is a benzyl isothiocyanate, such as 4-isothiocyanatobenzyl, 3-isothiocyanatobenzyl, 2-isothiocyanatobenzyl, wherein the benzyl isothiocyanate optionally has one or more alkyl substituents and/or one or more cyclic acid substituents, e.g., wherein the benzyl isothiocyanate optionally has substituents L1 and/or L2 at the benzylic position, wherein:
L1 is H or an alkyl or cyclic alkyl (when L1 is an alkyl or cyclic alkyl, the alkyl or cyclic alkyl can have carbon or heteroatom substitutions);
L2 is H or an alkyl or cyclic alkyl. (when L1 is an alkyl or cyclic alkyl, the alkyl or cyclic alkyl can have carbon or heteroatom substitutions); or
L1 and L2 form a cycle, such as a carbon cycle or a heterocycle or a fused cycle.
   33. The method of any of the foregoing embodiments, wherein the antibody or antigen binding fragment is conjugated to the chelator via a linker.
   34. The method of any of the foregoing embodiments, wherein the radioconjugate comprises the antibody or antigen binding fragment conjugated to 2-(4-isothiocyanatobenzyl)-1,4,7,10-tetraazacyclododecane-1,4,7,10-tetraacetic acid with the chelated actinium-225 (225 Ac), preferably, the 2-(4-isothiocyanatobenzyl)-1,4,7,10-tetraazacyclododecane-1,4,7,10-tetraacetic acid is covalently linked to one or more amino acid residues in the Fc region of the antibody.
   35. The method of any of the foregoing embodiments, wherein the radioconjugate comprises the antibody or antigen binding fragment conjugated to 2-S-(4-isothiocyanatobenzyl)-1,4,7,10-tetraazacyclododecane-1,4,7,10-tetraacetic acid with the chelated actinium-225 (225Ac), preferably, the 2-S-(4-isothiocyanatobenzyl)-1,4,7,10-tetraazacyclododecane-1,4,7,10-tetraacetic acid is covalently linked to one or more amino acid residues in the Fc region of the antibody.
   36. The method of any of the foregoing embodiments, wherein the radioconjugate comprises an average of from about 1 to about 4 chelator molecules conjugated to the antibody.
   37. The method of any of the foregoing embodiments, wherein the radioconjugate provides a specific activity from about 50 μCi to about 350 μCi, such as 50 μCi, 100 μCi, 150 μCi, 200 μCi, per about 2-3 mg of total antibody.
   38. The method of any of the foregoing embodiments, wherein the radioconjugate is administered in a pharmaceutical composition, preferably in a pharmaceutical composition formulated for intravenous administration, wherein the pharmaceutical composition comprises the radioconjugate and one or more pharmaceutically acceptable excipients.
   39. The method of any of the foregoing embodiments, wherein the pharmaceutical composition comprises the radioconjugate, a radioprotectant, surfactant and buffer, with a pH of 5-6.
   40. The method of any one of embodiments 38-39, wherein the pharmaceutical composition further comprises a conjugate comprising the antibody or the antigen binding fragment conjugated to the chelator without actinium-225 (225 Ac) chelated by the chelator.
   41. The method of any one of embodiments 38-40, wherein the pharmaceutical composition is administered with a total antibody mass of 2 to 10 mg per dose.
   42. The method of any one of embodiments 38-41, wherein the pharmaceutical composition is administered at 12.5 μCi/mL, 25 μCi/mL, 37.5 μCi/mL or 50 μCi/mL (at the time of dosing).
   43. The method of any one of the foregoing embodiments, wherein the radioconjugate or the pharmaceutical composition is administered to the patient intravenously.
   44. The method of any of the foregoing embodiments, wherein the patient:
1) has a prior treatment with at least one androgen receptor pathway inhibitor (ARPI);
2) can have a prior chemotherapy treatment;
3) has not had prior radioconjugate therapy; and/or
4) has no prior radiotherapy with >30% red marrow distribution.
   45. The method of any one of the foregoing embodiments, wherein the patient has received 0 to 12, for example 4, prior lines of therapy, such as ARPI and/or taxane.
   46. The method of any one of the foregoing embodiments, wherein the patient has met one or more, such as all, of the following criteria:
1) mCRPC with histologic confirmation of adenocarcinoma;
2) prior exposure to at least one androgen receptor-targeted therapy (eg, abiraterone acetate, enzalutamide, apalutamide, darolutamide), or prior taxane or other chemotherapy;
3) treatment with other agents for prostate cancer, if received, discontinued ≥2 weeks prior to first dose of the radioconjugate;
4) prior orchiectomy or medical castration; or, for patients who have not undergone orchiectomy, receiving ongoing androgen deprivation therapy with a gonadotropin releasing hormone (GnRH) analog (agonist or antagonist) prior to the first dose of the radioconjugate;
5) palliative radiotherapy (eg, soft tissue lesions) completed >2 weeks prior to the first dose of the radioconjugate, except for palliative radiotherapy for pain (eg, bone pain), which may be used any time prior to first dose;
6) Eastern Cooperative Oncology Group (ECOG) performance status of 0 or 1;
7) Hematology laboratory parameters within the following range:
   a. Hemoglobin >9.0 g/dL
   b. Absolute neutrophil count >1.5×10⁹/L
   c. Platelet count >100×10⁹/L
8) Clinical chemistry laboratory parameters within the following range before the first dose of study drug:
   a. Serum total bilirubin <1.5×upper limit of normal (ULN)
   b. Aspartate aminotransferase (AST) and alanine aminotransferase (ALT) ≤2.5×ULN (or ≤4×ULN for participants with tumor involvement in the liver)
   c. Calculated or measured creatinine clearance >50 mL/min/1.73 m² as determined by Modification of Diet in Renal Disease formula
   d. Grade ≤1 proteinuria. For proteinuria clearly attributable to other known medical conditions of the participant, such as hemorrhagic radiation cystitis or chronic hematuria caused by tumor infiltrates, Grade ≥1 proteinuria requires an approval from the sponsor's medical monitor.
      47. The method of any one of the foregoing embodiments, wherein the patient does not have one or more of the following:
1) Prior treatment with radium Xofigo® (Ra 223 dichloride), strontium, or samarium therapy or radioconjugate therapy;
2) Prior radiation therapy encompassing >30% of expected red marrow distribution;
3) Diffuse bone or bone-marrow involvement (ie, superscan—diffuse increased marrow uptake with reduced background and renal activity);
4) Spinal cord compression (unless treated, stable, and approved by the sponsor medical monitor);
5) Active CNS metastases;
6) Known history of myelodysplastic syndrome, leukemia, or hematological malignancy with features suggestive of myelodysplastic syndrome/acute myeloid leukemia at any timepoint;
7) Toxicity from prior anticancer therapy has not resolved to baseline levels or to Grade ≤1 (except alopecia, radiation tissue fibrosis, or peripheral neuropathy);
8) Malignancy diagnosis other than the disease under study within 2 years prior to the first dose of study drug, except for squamous and basal cell carcinoma of the skin, non-muscle invasive bladder cancer, or any malignancy considered cured or has minimal risk of recurrence within 1 year of first dose of study drug (in the opinion of both the investigator and sponsor's medical monitor);
9) Any systemic anti-neoplastic therapy (eg, chemotherapy, immunotherapy or biological therapy [including mAbs], poly ADP ribose polymerase [PARP] inhibitors)≤30 days prior to the first dose of the radioconjugate except for luteinizing hormone-releasing hormone agonists/antagonists or GnRH agonists/antagonists, and androgen axis drugs (e.g., enzalutamide, apalutamide, darolutamide, and/or abiraterone acetate plus prednisone) must have been discontinued ≥2 weeks prior to the first dose of the radioconjugate.
   48. The method of any one of the foregoing embodiments, wherein the treatment provides a lower rate of severe toxicities (toxicity of Grade 3 or higher according to the NCI CTCAE version 5.0), e.g., compared to the administering of the first dose of (i) at a fixed dose level and at a fixed dose schedule (e.g., compared to the administering of the first dose of (i) once every 8 weeks).
   49. The method of any one of the foregoing embodiments, wherein the treatment results in effective treatment of the prostate cancer, as measured by the PSA response, and/or Overall response rate (ORR) according to response criteria of Prostate Cancer Working Group 3 (PCWG3).
   50. The method of any one of the foregoing embodiments, wherein the prostate cancer is advanced prostate cancer, locally advanced prostate cancer, metastatic castration-sensitive prostate cancer (CSPC), metastatic castration-resistant prostate cancer (CRPC), non-metastatic CSPC, non-metastatic CRPC, or metastatic prostate cancer, preferably the prostate cancer is metastatic CRPC.
   51. The method of any one of embodiments 1-50, wherein (prior to the administering of a (therapeutically effective) first dose of a radioconjugate) the prostate cancer is metastatic hormone sensitive prostate cancer (mHSPC).
   52. The method of embodiment 51, wherein the metastatic hormone sensitive prostate cancer is metastatic hormone sensitive prostate cancer with non-castrate levels of testosterone (e.g., at study entry), for example with levels of testosterone >150 ng/dL.
   53. The method of embodiment 51 or 52, wherein the patient does not have undergone orchiectomy and is not receiving ongoing androgen deprivation therapy with a gonadotropin releasing hormone (GnRH) analog (agonist or antagonist) prior to the first dose of the radioconjugate.
   54. The method of any one of embodiments 51-53, wherein the mHSPC is a mHSPC with no (evidence of) metastasis to visceral organs.
   55. The method of any one of embodiments 51-54, wherein the mHSPC is a mHSPC with metastasis in no more than 5 (distinct) locations in the patient's body.
   56. The method of any one of embodiments 51-55, wherein the mHSPC is a mHSPC, which can be treated or which can be encompassed by no more than 5 radiation isocenters (i.e., no more than five radiation fields).
   57. The method of any one of embodiments 51-56, wherein the mHSPC is a mHSPC with metastasis in no more than 5 (distinct) locations in the patient's body, and the mHSPC is a mHSPC, which can be treated or which can be encompassed by no more than 5 radiation isocenters (i.e., no more than five radiation fields).
   58. The method of any one of embodiments 51-57, wherein the mHSPC is a mHSPC with no more than five (distinct) metastases (e.g., no more than five lesions on PSMA-PET scan).
   59. The method of any one of embodiments 51-58, wherein the mHSPC is a mHSPC with metastasis in no more than 5 (distinct) locations in the patient's body and with no (evidence of) metastasis to visceral organs.
   60. The method of any one of embodiments 51-59, wherein the mHSPC is a mHSPC with no more than five (distinct) metastases (e.g., no more than five lesions on PSMA-PET scan) and with no (evidence of) metastasis to visceral organs.
   61. The method of any one of claims 51-60, wherein the first dose of the radioconjugate administered in (i) is 150 μCi or 200 μCi, preferably 150 μCi.
   62. The method of any one of claims 51-61, wherein the patient has not received any dose of the radioconjugate prior to step (i), wherein the first dose of the radioconjugate administered in step (i) is 150 μCi or 200 μCi, preferably 150 μCi, and wherein the second dose of the radioconjugate administered in step (ii) is 100 or 150 μCi.
   63. The radioconjugate as defined in any one of the foregoing embodiments for use in the method of any one of the preceding embodiments.
   64. The pharmaceutical composition as defined in any one of embodiments 38-50 for use in the method of any one of embodiments 38-50.
   65. The pharmaceutical composition as defined in any one of embodiments 38-62 for use in the method of any one of embodiments 38-60.
   66. The pharmaceutical composition as defined in any one of embodiments 13, 15 or 21 for use in the method of any one of embodiments 13, 15 or 21.
   67. The pharmaceutical composition as defined in any one of embodiments 51-62 for use in the method of any one of embodiments 51-62.
   68. A product comprising the radioconjugate as defined in any one of the preceding embodiments for use in the method of any one of the preceding embodiments.

Various embodiments or subject matter have been described. It will be understood that various modifications can be made without departing from the spirit and scope of the invention. Accordingly, the following examples are intended to illustrate but not limit the scope of inventions described in the claims.

### EXAMPLES

The following examples are provided to further describe some of the embodiments disclosed herein. The examples are intended to illustrate, not to limit, the disclosed embodiments.

### Example 1: Manufacture of ²²⁵Ac-DOTA-h11B6 and Formulations Containing ²²⁵Ac-DOTA-h11B6

This example describes manufacture of drug product containing ²²⁵Ac-DOTA-h11B6. Other manufacturing methods can also be used.

This example describes processes for the manufacture of ²²⁵Ac-DOTA-h11B6 drug product and a solution containing the drug product. The antibody h11B6 can be prepared as described, for example, in U.S. Pat. No. 10,100,125, which is incorporated by reference herein. The h11B6 antibody of the present invention can comprise a heavy chain constant region comprising the amino acid sequence of SEQ ID NO: 9, and/or a light chain constant region comprising the amino acid sequence of SEQ ID NO: 10.

The antibody h11B6 can also be prepared using methods described in U.S. Pat. No. 9,873,891, which is incorporated by reference herein, using a CHO DG44 derived cell line and an hEF1α promoter double gene vector, commercially available from Fujifilm Diosynth Biotechnologies.

Following preculture and expansion, the cell culture can be clarified using known filtration techniques. The filtrate is concentrated and diafiltered to a target final concentration of 10 g/L in buffer (25 mM NaOAc, pH 5.5). The h11B6 is filtered through a 0.2-μm filter, filled into sterilized bags, and can be frozen at ≤−65° C. for long-term storage, prior to conjugation.

The thawed h11B6 is then diafiltered (buffer exchanged) to 50 mM Bicine, 120 mM NaCl, pH 8.5 for the subsequent conjugation of DOTA (1,4,7,10-tetraazacyclododecane-1,4,7,10-tetraacetic acid) to h11B6. The retentate from the prior step is transferred to a reactor and stirred while warming to 25° C. A solution of p-SCN-Bn-DOTA in water is prepared and added to the reactor. The reaction is maintained at 25° C. for 20 hours. The product of the conjugation reaction (DOTA-h11B6) is transferred directly to the retentate vessel for the final diafiltration with 25 mM NaOAc, pH 5.5. Next, DOTA-h11B6 conjugate intermediate is filtered through a 0.2-μm filter, filled into sterilized polycarbonate containers, and can be frozen at ≤−65° C. for long-term storage.

The conjugation reaction results in addition of multiple DOTA molecules to the epsilon amino group of lysine side chains of the h11B6 mAb. The conjugate-to-antibody ratio (CAR), which designates the number of DOTA molecules per h11B6 mAb molecule, can be measured by intact mass analysis using RP-HPLC with online mass analysis. Based on the molecular structure of p-SCN-Bn-DOTA, each DOTA residue adds 552 Da mass to the antibody, which can be readily detected by intact mass analysis. To reduce the sample complexity, DOTA-h11B6 conjugate intermediate was treated with PNGase F to remove N-linked glycans and carboxypeptidase B to remove C-terminal lysine residues.

The ²²⁵Ac-DOTA-h11B6 drug product is produced in a continuous operation from the precursor, DOTA-h11B6 conjugate intermediate, which reacts with ²²⁵Actinium trichloride to generate an ²²⁵Actinium-radiolabeled drug substance with a target specific activity of ≥170 μCi/mg. The ²²⁵Ac-DOTA-h11B6 drug substance is synthesized, purified by PD-10 column purification, and formulated in situ. The four drug product presentations (50, 100, 150, and 200 μCi in 2 mg protein) are manufactured by blending of the ²²⁵Ac-DOTA-h11B6 drug substance with DOTA-h11B6 and reformulation buffer, as described below. The blended product presentations are individually sterile filtered and aseptically filled into the final patient vial.

An intermediate purification buffer (acetate buffer, radioprotectant, surfactant, pH 5.5) is prepared for the final compounded purification buffer and can be stored at 2-8° C. for ≤30 days prior to use. Sodium ascorbate is added to the intermediate purification buffer and filtered through a 0.2-μm sterilizing filter into a sterile product holding vessel to produce the final compounded purification buffer (acetate buffer, radioprotectant, surfactant, pH 5.5).

The DOTA-h11B6 conjugate intermediate is thawed at room temperature. A solution of actinium trichloride is prepared by dissolving actinium trinitrate in 0.1 N hydrochloric acid (it is also possible to use sources of Ac-225 that are already in trichloride form). Actinium trichloride (800-1300 μCi) is incubated with 4.4 mg DOTA-h11B6 and sodium acetate buffer (pH adjustment with acetic acid, prepared ahead of time and stored ≤6 months), pH adjusted to 6.5. ²²⁵Ac-DOTA-h11B6 is then purified on a PD-10 column pre-conditioned and eluted with the final purification buffer. After purification, the amount of radioactivity is measured.

In preparation to achieve the four dose amounts (50, 100, 150, and 200 μCi), the DOTA-h11B6 conjugate intermediate is reformulated to 0.5 mg/mL (acetate buffer, radioprotectant, surfactant, pH 5.5) using the final reformulation buffer and filtered through a 0.2-μm sterilizing filter. ²²⁵Ac-DOTA-h11B6 is then dispensed into an intermediate vial to achieve the desired unit dose (50, 100, 150, and 200 μCi in 4 mL at the anticipated time of administration to a patient) and reformulated DOTA-h11B6 conjugate intermediate is added to a volume of 6.8 mL to produce the drug product. The drug product is then filtered through a 0.2-μm sterilizing filter and aseptically filled to a volume of 4.8 mL. The remaining drug product is also filtered through a 0.2-μm sterilizing filter and aseptically filled for release testing of the drug product. The drug product is immediately stored at 2-8° C.

Thus, the radiolabeled drug product ²²⁵Ac-DOTA-h11B6 is prepared as a sterile solution for intravenous injection and does not contain a preservative. The ²²⁵Ac-DOTA-h11B6 is available in four drug product (DP) unit doses: 50, 100, 150 and 200 μCi at anticipated time of administration to a patient.

### Example 2: Use of an Actinium-225-Labeled Antibody Targeting Human Kallikrein-2 (hK2) for Advanced Prostate Cancer

This example describes a first-in-human Phase 1 study to evaluate the safety, pharmacokinetics, pharmacodynamics, and preliminary antitumor activity of ²²⁵Ac-DOTA-h11B6 administered to adult patients with mCRPC who have disease progression on or following AR-targeted therapy. A formulation containing ²²⁵Ac-DOTA-h11B6 in a buffer, one or more radioprotectants, and surfactant, pH 5.5 was administered to patients in the Phase 1 trial. As discussed herein, ²²⁵Ac-DOTA-h11B6 is an hK2-specific monoclonal antibody, h11B6, labeled with DOTA and chelated to the α-particle-emitting radionuclide ²²⁵Ac, and is a radioimmunotherapy targeted to the hK2 antigen.

The primary objectives were to determine the safety and recommended Phase 2 dose(s) (RP2Ds) of ²²⁵Ac-DOTA-h11B6 and to evaluate the incidence, duration, and severity of adverse events, including dose-limiting toxicity (DLT). Secondary objectives and endpoints evaluate the preliminary antitumor activity and provide a further understanding of the pharmacology of ²²⁵Ac-DOTA-h11B6. See, e.g., Table 2.

TABLE 2
Objectives and Endpoints of Phase 2

| | |
|---|---|
| Objectives | Endpoints |

Primary

| | |
|---|---|
| Part 1 (Dose Escalation) | Part 1 (Dose Escalation) |
| Determine RP2Ds of ²²⁵Ac- | Incidence, duration, and severity of adverse |
| DOTA-h11B6 | events, including dose-limiting toxicity |
| Part 2 (Dose Expansion) | Part 2 (Dose Expansion) |
| Determine safety at | Incidence and severity of adverse events |
| the RP2D(s) | |

Secondary

| | |
|---|---|
| Assess the preliminary | PSA response |
| antitumor activity | Overall response rate (ORR) according to |
| | response criteria of Prostate Cancer |
| | Working Group 3 (PCWG3) |
| Assess the | Serum radioactivity-time profiles |
| pharmacokinetics | and pharmacokinetic parameters |
| and immunogenicity | for ²²⁵Ac-DOTA-h11B6 |
| | Presence of anti-²²⁵Ac-DOTA-h11B6 |
| | antibodies |

Exploratory

Explore the relationships between pharmacokinetics, pharmacodynamics,
adverse event profile, and antitumor activity.
Rate of PSA decline following single-dose and radiographic response.

²²⁵Ac-DOTA-h11B6 was administered to adult males ≥18 years with mCRPC who had had prior exposure to at least one novel AR-targeted therapy. Administration of ²²⁵Ac-DOTA-h11B6 was conducted in 2 parts: dose escalation (Part 1) and dose expansion (Part 2).

Response to treatment was assessed according to the response criteria of PCWG3.

Blood samples were collected to characterize the pharmacokinetics of serum radioactivity and the concentration of h11B6 antibody, and to characterize the presence of anti-drug antibodies of ²²⁵Ac-DOTA-h11B6.

The safety of ²²⁵Ac-DOTA-h11B6 was assessed by physical examinations, Eastern Cooperative Oncology Group (ECOG) performance status, electrocardiograms, clinical laboratory tests, vital signs, and adverse event monitoring. Echocardiogram or multigated acquisition scans were assessed at screening; subsequent evaluations were conducted if clinically indicated. The severity of adverse events was assessed using National Cancer Institute Common Terminology Criteria for Adverse Events (Version 5.0). Concomitant medication usage will be recorded.

Dose escalation decisions were supported by a modified continual reassessment method (mCRM) based on a Bayesian logistic regression model (BLRM) with overdose control (EWOC).

Inclusion Criteria include the following:

Each potential patient must satisfy all of the following criteria:

| | |
|---|---|
| 1 | Histologic: mCRPC with histologic confirmation of adenocarcinoma. |
| | Adenocarcinoma with small-cell or neuroendocrine features is allowed |
| 2 | Must have had prior exposure to at least one novel androgen receptor (AR) |
| | targeted therapy (example, abiraterone acetate, enzalutamide, apalutamide, |
| | darolutamide); prior taxane or other chemotherapy is acceptable but not required |
| 3 | Treatment with other agents for prostate cancer, if received, must have been |
| | discontinued greater than or equal to (≥) 2 weeks prior to first dose. |
| 4 | Adequate organ functions as reflected in laboratory parameters. |
| 5 | ECOG performance status of 0 or 1 |

Exclusion Criteria include the following:

Any potential patient who meets any of the following criteria will be excluded:

| | |
|---|---|
| 1 | Part 1: Prior treatment with radium Xofigo (Ra 223 dichloride), strontium, or |
| | samarium therapy or radioconjugate therapy |
| 2 | Known history of myelodysplastic syndrome, leukemia, or hematological |
| | malignancy with features suggestive of myelodysplastic syndrome/acute myeloid |
| | leukemia at any timepoint |
| 3 | Toxicity from prior anticancer therapy has not resolved to baseline levels or to |
| | Grade less than or equal to ≤1 (except alopecia, radiation tissue fibrosis, or |
| | peripheral neuropathy) |
| 4 | Known allergies, hypersensitivity, or intolerance to ²²⁵Ac-DOTA-h11B6 or its |
| | excipients and protein therapeutics |
| 5 | Active or chronic hepatitis B or hepatitis C infection |

### A. Part 1: Dose Escalation

Participants received intravenous (IV) injection of ²²⁵Ac-DOTA-h11B6 with one or multiple doses at the amounts described below.

In Part 1, 50 μCi/2 mg ²²⁵Ac-DOTA-h11B6 was administered to the first dose escalation cohort. After the DLT evaluation in this initial cohort had been conducted, dose escalation to the next dose level of radioactive ²²⁵Ac-DOTA-h11B6 was based on the review of all available additional data including, but not limited to, pharmacokinetic, pharmacodynamic, safety, and preliminary antitumor activity.

There are two components to the final drug product that will be administered to participants in this study: the ²²⁵Ac-DOTA-h11B6 and the unlabeled DOTA-h11B6 antibody. The two components can be pre-mixed in a single vial. The two components will be provided for each participant visit at the prescribed radioactivity dose, and a total antibody mass amount of between 2 and 10 mg. See, Table 3.

TABLE 3
²²⁵Ac-DOTA-h11B6 Radiotherapy Administration

| | | |
|---|---|---|
| Study drug: | ²²⁵Ac-DOTA-h11B6 (Radiolabeled | Naked h11B6 antibody (Non- |
| | Monoclonal Antibody with | radiolabeled Antibody with |
| | DOTA Linker) | DOTA Linker) |
| Route of | IV injection | IV injection |
| administration | | |
| Unit dose | 50, 100, 150, or 200 μCi per 4 mL with | 10 mg/mL |
| strength(s)/ | protein concentration of 2 mg/4 mL. | |
| Dosage levels | Doses above 200 μCi will have a total | |
| | volume of 8 mL with protein | |
| | concentration of 2 mg/4 mL. | |
| Dosage | Refrigerated liquid | Refrigerated liquid |
| formulation | | |

| | |
|---|---|
| Schedule of | Once every 8 weeks for up to 4 doses; additional doses |
| administration | can be considered after discussion with the sponsor. |
| Dosing | The DOTA-mAb and radioconjugate ²²⁵Ac-DOTA-h11B6 can be pre- |
| instructions | mixed in a single vial. The two components will be provided |
| | for each participant visit at the prescribed radioactivity |
| | dose, and a total antibody mass amount of between 2 and 10 mg. |

The 225Ac-DOTA-h11B6 radioactive investigational product is a single use, sterile, refrigerated solution for injection in a cyclic olefin polymer vial closed with a latex free stopper and aluminum seal. The 225Ac-DOTA-h11B6 was formulated in acetate in sterile water at pH 5.5. The investigational product was clear, colorless to slightly yellow, and free of visible particulate matter. The 225Ac-DOTA-h11B6 vials were stored refrigerated in the temperature range of 2-8° C. and protected from light. The drug product did not contain any preservatives and was designed for single-use only. The vial supplied to the clinic contains an overfill of 0.8 mL (total 4.8 mL) to allow a final dose withdrawal of 4.0±0.4 mL, dependent upon the actual time of administration.

Dose escalation was supported using an adaptive dose escalation strategy guided by the modified continual reassessment method based on a BLRM with EWOC.

The RP2D(s) will be determined after review of all available pharmacokinetic, pharmacodynamic, safety, and efficacy data. Once the RP2D(s) have been determined, patients will be treated to confirm the safety, pharmacokinetics, pharmacodynamics, and preliminary antitumor activity of ²²⁵Ac-DOTA-h11B6 at the RP2D(s) in Part 2.

### B. Part 2: Dose Expansion

In Part 2, the RP2D(s) of ²²⁵Ac-DOTA-h11B6, as determined in Part 1, will be administered to patients in one or more cohorts.

All adverse events and adverse events fulfilling the criteria of DLT, will be reviewed and confirmed. Adverse events will be evaluated according to NCI CTCAE Version 5.0. Criteria for DLT are outlined in Table 4.

TABLE 4
Dose-limiting Toxicity Criteria ^{a}
Hematologic Toxicity

| | |
|---|---|
| Neutrophil count decreased | Febrile neutropenia |
| | Neutropenia: Grade 4 for >5 days |
| Platelet count decreased | Grade ≥3 thrombocytopenia with bleeding or Grade 4 |
| | thrombocytopenia of any duration |
| Any hematological toxicity | Grade 5 |

Non-hematological Toxicity

Any non-hematological toxicity of Grade ≥3, except for the following ^{c}:
Grade 3 fatigue, fever, constipation, or diarrhea lasting <7 days with best supportive care
Grade 3 nausea or vomiting lasting ≤48 hours that resolves to Grade ≤1 either spontaneously
or with best supportive care
Grade ≥3 ALT or AST that resolves to Grade ≤1 or baseline within 7 days, unless criteria for
Hy's law are met ^{b}
Isolated Grade ≥3 ALP ^{a} or GGT increase that returns to Grade ≤1 or baseline within 7 days
Grade ≥3 lipase or amylase increase not associated with clinical or radiological evidence of
pancreatitis
Grade ≥3 electrolyte abnormalities ^{c} that last ≤72 hours resolving spontaneously or with best
supportive care
^{a} Unless unequivocally due to the underlying malignancy or an extraneous cause.
^{b} Hy's Law criteria defined as ALT or AST value ≥3 × ULN, total bilirubin ≥2 × ULN, and ALP ≤2 × ULN; with no alternative etiology. For patients with baseline Grade 2 elevation of AST or ALT due to liver metastasis, ALT or AST >3 × baseline or AST or ALT >8 × ULN, whichever is lower, combined with total bilirubin >2 × baseline and >2 × ULN will be considered meeting Hy's law.
^{c} Any chemistry abnormalities Grade ≥3 occurring during the DLT period must be reassessed to confirm
the grade and resolution to Grade ≤2.

Outcome measures are provided in Table 5.

TABLE 5
Outcome Measures

| Outcome Measure | Time Frame | Description |
|---|---|---|
| Part 1 and | Up to 2 | An AE is any untoward medical occurrence in a |
| Part 2: Number | years and | patient that does not necessarily have a causal |
| of patients | 4 months | relationship with the pharmaceutical/biological agent. |
| with Aes as a | | |
| measure of safety | | |
| and tolerability | | |
| Part 1: Number | Up to 2 | Number of patients with DLT will be assessed. The |
| of patients | years and | DLTs are specific adverse events and are defined as |
| with DLT | 4 months | any of the following: high grade non-hematologic |
| | | toxicity, or hematologic toxicity. |
| Part 1 and | Up to 2 | Severity will be graded according to the NCI CTCAE |
| Part 2: Number | years and | version 5.0. Severity scale ranges from Grade 1 (Mild) |
| of patients | 4 months | to Grade 5 (Death). Grade 1 = Mild, Grade 2 = |
| with Aes by | | Moderate, Grade 3 = Severe, Grade 4 = Life- |
| severity | | threatening and Grade 5 = Death related to |
| | | adverse event. |

Secondary outcome measures are provided in Table 6.

TABLE 6
Secondary Outcome Measures

| Outcome Measure | Time Frame | Description |
|---|---|---|
| Percentage of patients | Week 12 | PSA response rate is defined as the |
| with PSA response | | percentage of patients with a decline of |
| | | PSA of 50% or more from baseline at |
| | | Week 12. |
| Overall Response | Up to 2 | ORR is defined as the percentage of |
| Rate (ORR) | years and | patients who have a partial response (PR) |
| | 4 months | or better according to Response |
| | | Evaluation Criteria in Solid Tumors |
| | | (RECIST) version 1.1 without evidence of |
| | | bone progression according to PCWG3. |
| Cₘₐₓ of ²²⁵Ac-DOTA-h11B6 | Up to 2 | Cmax is defined as the maximum |
| | years and | observed serum |
| | 4 months | concentration/radioactivity of ²²⁵Ac- |
| | | DOTA-h11B6. |
| Tₘₐₓ of ²²⁵Ac-DOTA-h11B6 | Up to 2 | Tmax is defined as time to reach |
| | years and | maximum observed serum |
| | 4 months | concentration/radioactivity of ²²⁵Ac- |
| | | DOTA-h11B6. |
| AUC₀₋ₜ of ²²⁵Ac-DOTA-h11B6 | Up to 2 | AUC₀₋ₜ is defined as the area under the |
| | years and | serum concentration-time curve from time |
| | 4 months | zero to t of ²²⁵Ac-DOTA-h11B6. |
| Number of patients with | Up to 2 | Number of patients with anti-²²⁵Ac- |
| anti-²²⁵Ac-DOTA-h11B6 | years and | DOTA-h11B6 antibodies will be assessed |
| antibodies | 4 months | to evaluate the potential immunogenicity. |

### Interim Clinical Results

Patients (pts) received ²²⁵Ac-DOTA-h11B6 escalating from 50 to 300 μCi IV with doses repeating every 8-12 weeks. Additional patients received a maximum of 2 doses of 250 μCi IV with a dosing interval of 8-12 weeks or a single dose of 400 μCi IV. Additional patients received a single dose of 250 μCi IV with the plan to administer subsequent doses adaptively (with the adaptive dosing plan described below). In total 75 pts received ≥1 dose of ²²⁵Ac-DOTA-h11B6.

Prolonged clinical, biochemical, and radiographic response was noted starting with the 150 μCi dose. Exposure and efficacy for all 75 patients treated with ²²⁵Ac-DOTA-h11B6 are summarized in Table 7 below, e.g., 46/75 (61.3%) of the patients experienced grade ≥3 TEAEs, and 12 (16%) had a serious treatment-related TEAE. TEAEs of note included thrombocytopenia (58.7%) and interstitial lung disease (7%, ILD; all occurred at cumulative doses ≥500 μCi and prior to implementation of pulmonary function surveillance). Grade ≥3 TEAEs (≥10%) included anemia (25.3%), thrombocytopenia (17.3%), lymphopenia (14.7%), and leukopenia (8%). Nine (12.0%) pts discontinued treatment due to TRAEs; 4 patients died a result of TRAEs. PSA50 rate was 45.1%. To date, across all dose cohorts, 29 pts (39%) remained on treatment for ≥6 months, including pts who have been on treatment for over 2 years.

In this FIH study, key TEAEs of ²²⁵Ac-DOTA-h11B6 were thrombocytopenia and ILD were associated with cumulative dose. At ≥150 μCi, 1-2 doses of 225Ac-DOTA-h11B6 elicited durable biochemical and radiographic responses. Evaluation of the preliminary RP2D is ongoing. These data highlight the promise of both α-emitting TRT and hK2 as a novel target.

TABLE 7

| | All doses and schedules |
|---|---|
| Parameter, | 225Ac-DOTA-h11B6 |
| median (range) or n (%) | (n = 75) |
| No. of prior therapies | 4 (0-12) |
| Confirmed objective response rate^{a} | 3 (8.6)^{a} |
| Complete response | 1 (2.9) |
| Partial response | 2 (5.9) |
| Disease control rate at 6 months^{b} | 21 (28.0) |
| PSA50^{c} | 32 (45.1) |
| PSA90 | 8 (11.3) |

^{a}In pts with measurable disease at baseline (n = 34).
^{b}Proportion alive and radiographic progression free for ≥6 months from the start of treatment.
^{c}Pts who were on treatment for >=12 weeks/discontinued treatment/achieved any PSA50.

As of October 2024, 66 patients were dosed on scheduled (Q8-12W) intervals in Part 1 at doses ranging from 50-400 μCi and 46 patients received GRID. Across all dose levels, the most frequent (≥20%) AEs observed were (% [% G3+]): thrombocytopenia (58 [14]); fatigue (56 [2]); anemia (44 [22]); decreased appetite (40 [3]); nausea (38 [2]); leukopenia (35 [11]); diarrhea (24 [1]); vomiting (24 [2]); dizziness (24 [0]); dry mouth (23 [0]); dyspnea (22 [0]); and lymphopenia (21 [14]). Notable toxicities observed with cumulative doses ≥500 μCi on a Q8-12W schedule included interstitial lung disease (8%, including 2 fatal cases) and persistent G3/G4 thrombocytopenia (~20%). After implementation of enhanced pulmonary monitoring and GRID dosing, only 2 non-serious ILD cases were observed (G1 pneumonitis; G2 hypoxia) and there have been no cases of persistent thrombocytopenia to date. Durable PSA50 responses were observed in ~50% of patients across all dose cohorts. Objective responses were seen in 21% of RECIST-evaluable patients (including visceral metastases) in the 250 μCi initial dose level cohorts.

Interim results demonstrate efficacy and durability in advanced mCRPC.

### Adaptive Dosing Plan

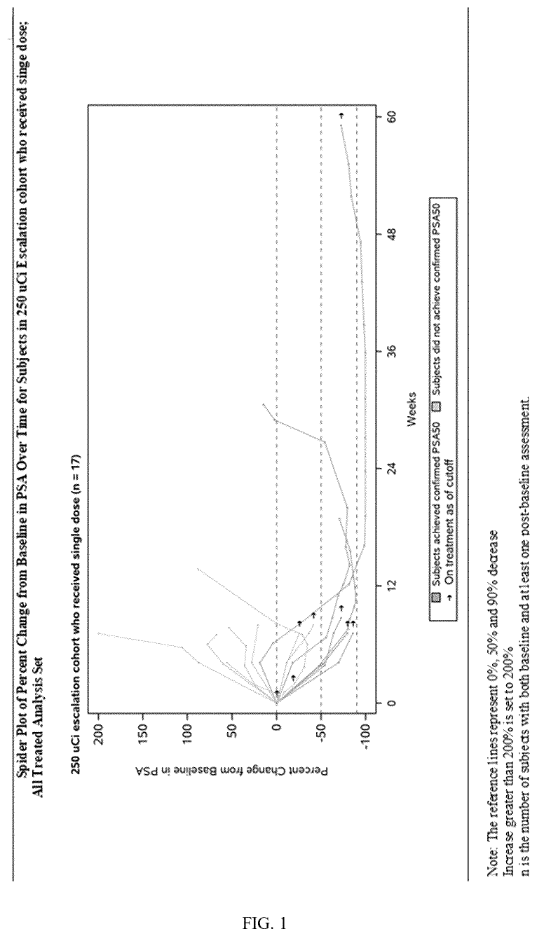

An adaptive dosing plan was implemented to optimize safety and efficacy. This dosing plan was supported by observed efficacy in patients who received only a single dose of 250 μCi IV (FIG. 1). PSA responses of 4 months to >1 year were observed, suggesting that disease control provided by a single dose could extend beyond the 8-12 week dosing interval. In addition, the frequency of grade 3 thrombocytopenia and anemia was lower after a single dose than in the overall population of patients (5.9% grade 3 thrombocytopenia and 11.8% grade 3 anemia). The adaptive dosing plan was as follows:

For new participants to the study, the first dose of the adaptive dosing plan was 250 μCi. Patients who were already enrolled and dosed in the study were also enrolled in the adaptive dosing plan. For example, patients having already received 1 dose of 400 μCi; or 3 doses of 150 μCi; or 2 doses of 250 μCi; or 1 dose of 200 μCi, 1 dose of 150 μCi and 1 dose of 150 μCi) were also enrolled in the study.

Subsequent doses were scheduled only once the following conditions are met:
absence of PSA progression (PSA level ≥25% and ≥2 ng/mL above pretreatment baseline) for 12 weeks since last dose;
PSA progression (PSA level ≥25% and ≥2 ng/ml above Nadir) at more than 12 weeks since last dose; and
platelet count above 100×10E9 par L.

Nadir is defined as the lowest PSA measured after the prior dose (including the PSA measured immediately prior to that dose).
Dosage for subsequent doses: will be between 100-250 μCi; for example
For participants with cumulative exposure ≤450 μCi, dosage is 150 μCi
   Includes participants who have received a single dose of 250 μCi or 400 μCi
   Also includes participants who have received 250 μCi followed by 150 μCi
For participants with cumulative exposure of 500 μCi, dosage is 100 μCi
   Includes participants who have received 2 doses of 250 μCi

Table 8 shows some examples of dosages for subsequent doses in the adaptive dosing plan.

TABLE 8

| Prior doses | Cumulative dose to date | Next dose |
|---|---|---|
| 250 μCi × 1 | 250 μCi | 150 μCi |
| 400 μCi × 1 | 400 μCi | 150 μCi |
| 150 μCi × 3 | 450 μCi | 150 μCi |
| 250 μCi × 2 | 500 μCi | 100 μCi |
| 200 μCi + 150 μCi + | 500 μCi | 100 μCi |
| 150 μCi | | |

Visit and Evaluation Schedule after Day 50 (Adaptive Dosing Plan)

### Every 4 Weeks

Physical exam
SpO2 (resting and ambulatory)
Safety labs (hematology and chemistry)
PSA

### After 6 Months

Safety assessments (exam, SpO2, labs) can be every 12 weeks
PSA to continue every 4 weeks

### Disease Evaluation Scans

Every 8 (+1) weeks after the first dose of study drug for the first 24 weeks then every 12 (+1) weeks thereafter
   As of December 2024, subsequent doses were given to some patients with rising PSA and resulted in achieving PSA50 again.

### Example 3: a Patient Treated with Adaptive Dosing

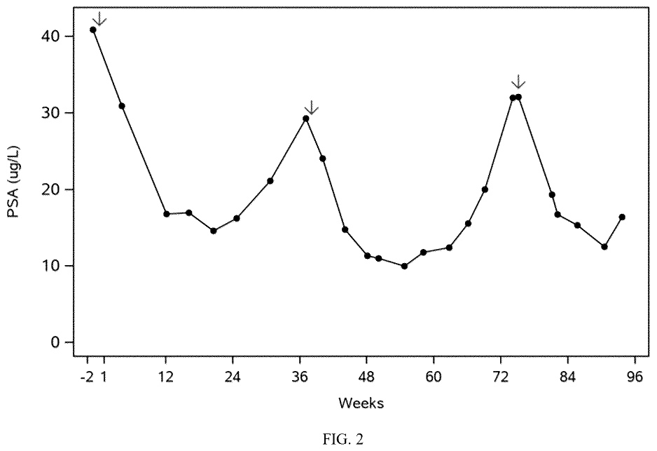

A patient received an initial dose of 200 μCi IV and had a PSA response lasting beyond 12 weeks. The patient was re-dosed with a lower dose of 150 μCi IV at 38 weeks following PSA progression and with a platelet count >1×10e9/L. The PSA again responded, and the patient received a third dose with 150 μCi IV approximately 38 weeks later, again with PSA response. The overall duration of disease control is approaching 2 years, and the patient has experienced no grade ≥3 toxicities (FIG. 2).

### Example 4: Metastatic Hormone Sensitive Prostate Cancer (Metastatic HSPC) Patients

The Phase 1 study of Example 2 is implemented in metastatic HSPC patients, including oligometastatic HSPC patients (Part 4 of the study). The objectives and endpoints are the same as in example 2. In Part 4, the RP2D(s) of ²²⁵Ac-DOTA-h11B6, as determined in Part 1, is administered to the mHSPC patients (as described in Example 2 for the mCRPC patients of Part 2). The Adverse Events and Outcomes of Part 4 are identical to those described in Example 2 for Part 2. Part 4 implements the Adaptive Dosing Plan as described in Example 2.

Part 4 comprises:
Cohort 4a: patients with metastatic HSPC; and
Cohort 4b: patients with oligometastatic HSPC.

The patients of Cohort 4b receive stereotactic body radiation therapy (SBRT) prior to the first dose of ²²⁵Ac-DOTA-h11B6 (e.g., 14-56 days prior to the first dose of ²²⁵Ac-DOTA-h11B6). Oligometastatic HSPC refers to a subgroup of metastatic HSPC with a limited number of metastases, typically with no metastasis to visceral organs, more particularly a metastatic HSPC which can be treated or encompassed by no more than five radiation fields and with no metastasis to visceral organs and, typically a metastatic HSPC with metastasis in no more than five (distinct) locations in the patient's body and with no metastasis to visceral organs.

The patients of Cohorts 4a and 4b receive the first dose of ²²⁵Ac-DOTA-h11B6 as described in Example 2 (250 μCi administered by IV injection), and any subsequent dosing of ²²⁵Ac-DOTA-h11B6 is decided and determined in accordance with the Adaptive Dosing Plan. Alternatively, the patients of Cohorts 4a and 4b receive the first dose of 225Ac-DOTA-h11B6 as described in Example 2 but at dose level lower than 250 μCi (150 μCi administered by IV injection), and any subsequent dosing of 225Ac-DOTA-h11B6 is decided and determined in accordance with the Adaptive Dosing Plan

Inclusion criteria for Cohorts 4a and 4b include:
metastatic HSPC with non-castrate levels of testosterone (>150 ng/dL);
must be ineligible for or unwilling to take Androgen Deprivation Therapy or other Androgen Receptor-targeted therapy at this time (e.g., at the time of enrollment);
in addition: for Cohort 4b:
   1) must have recurrent disease after definitive treatment to the prostate that is metastatic either to bone, lymph node, or both without evidence of either measurable or non-measurable metastasis to visceral organs; and
   2) must have oligometastatic disease that can be encompassed in ≤5 radiation fields

Patients of Part 4 are at an earlier stage of the disease than patients of Part 2. In the Study, patients of Part 4 have not undergone orchiectomy and have not been receiving ongoing androgen deprivation therapy with a gonadotropin releasing hormone (GnRH) analog (agonist or antagonist) prior to the first dose of ²²⁵Ac-DOTA-h11B6.

Exclusion criteria for Cohorts 4a and 4b include that the patients must not have received ADT or AR-targeted therapy less than or equal to 56 days prior to the first dose of ²²⁵Ac-DOTA-h11B6.

SEQUENCE LISTING

| SEQ ID NO: | Sequence |
|---|---|
| 1 | SDYAWN |
| 2 | YISYSGSTTYNPSLKS |
| 3 | GYYYGSGF |
| 4 | KASESVEYFGTSLMH |
| 5 | AASNRES |
| 6 | QQTRKVPYT |
| 7 | QVQLQESGPGLVKPSDTLSLTCAVSGNSITSDYAWNWIRQPPGKGLEWI |
| | GYISYSGSTTYNPSLKSRVTMSRDTSKNQFSLKLSSVTAVDTAVYYCAT |
| | GYYYGSGFWGQGTLVTVSS |
| 8 | DIVLTQSPDSLAVSLGERATINCKASESVEYFGTSLMHWYQQKPGQPPK |
| | LLIYAASNRESGVPDRFSGSGSGTDFTLTISSLQAEDVAVYYCQQTRKV |
| | PYTFGQGTKLEIK |
| 9 | ASTKGPSVFPLAPSSKSTSGGTAALGCLVKDYFPEPVTVSWNSGALTSG |
| | VHTFPAVLQSSGLYSLSSVVTVPSSSLGTQTYICNVNHKPSNTKVDKKV |
| | EPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQD |
| | WLNGKEYKCKVSNKALPAPIEKTISKAKGQPREPQVYTLPPSREEMTK |
| | NQVSLTCLVKGFYPSDIAVEWESNGQPENNYKTTPPVLDSDGSFFLYSK |
| | LTVDKSRWQQGNVFSCSVMHEALHNHYTQKSLSLSPGK |
| 10 | RTVAAPSVFIFPPSDEQLKSGTASVVCLLNNFYPREAKVQWKVDNALQ |
| | SGNSQESVTEQDSKDSTYSLSSTLTLSKADYEKHKVYACEVTHQGLSSP |
| | VTKSFNRGEC |
| 11 | QVQLQESGPGLVKPSDTLSLTCAVSGNSITSDYAWNWIRQPPGKGLEWI |
| | GYISYSGSTTYNPSLKSRVTMSRDTSKNQFSLKLSSVTAVDTAVYYCAT |
| | GYYYGSGFWGQGTLVTVSSASTKGPSVFPLAPSSKSTSGGTAALGCLV |
| | KDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFP |
| | PKPKDTLMISRTPEVTCVVVDVSHEDPEVKFNWYVDGVEVHNAKTKP |
| | REEQYNSTYRVVSVLTVLHQDWLNGKEYKCKVSNKALPAPIEKTISKA |
| | KGQPREPQVYTLPPSREEMTKNQVSLTCLVKGFYPSDIAVEWESNGQPE |
| | NNYKTTPPVLDSDGSFFLYSKLTVDKSRWQQGNVFSCSVMHEALHNH |
| | YTQKSLSLSPGK |
| 12 | DIVLTQSPDSLAVSLGERATINCKASESVEYFGTSLMHWYQQKPGQPPK |
| | LLIYAASNRESGVPDRFSGSGSGTDFTLTISSLQAEDVAVYYCQQTRKV |
| | PYTFGQGTKLEIKRTVAAPSVFIFPPSDEQLKSGTASVVCLLNNFYPREA |
| | KVQWKVDNALQSGNSQESVTEQDSKDSTYSLSSTLTLSKADYEKHKV |
| | YACEVTHQGLSSPVTKSFNRGEC |

1. A method of treating prostate cancer, in a patient in need thereof, comprising:
(i) administering to the patient a therapeutically effective first dose of a radioconjugate comprising an antibody or an antigen binding fragment having binding specificity for hK2 conjugated to a chelator, optionally via a linker, wherein actinium-225 (²²⁵Ac) is chelated to the chelator; wherein no prostate-specific antigen (PSA) progression relative to a serum PSA level of the patient measured prior to the administering of (i) is observed for a period of time after the administering of the first dose; and
(ii) administering to the patient a therapeutically effective second dose of the radioconjugate after the period of time of (i), if the following conditions are met before the administering of the second dose:
a. there is a PSA progression after the period of time of (i), relative to the lowest serum PSA level of the patient from immediately prior to the administering of (i) to the end of the period of time; and
b. the patient has a platelet count above 100×10E9 per liter of blood;
(iii) optionally repeating (ii).

2. (canceled)

3. The method of claim 1, wherein the period of time in (i) is 12 weeks or more than 12 weeks.

4. The method of claim 1, further comprising monitoring the serum PSA level of the patient one or more times after the administering of step (i), and optionally further comprising measuring or obtaining the measurement of the serum PSA level of the patient immediately before the administering of (i).

5. The method of claim 1, wherein the second dose of the radioconjugate of (ii) is not administered to the patient, if there is no PSA progression relative to the lowest serum PSA level of the patient, and/or if the patient has a platelet count of 100×10E9 per liter of blood or lower.

6. The method of claim 5, wherein there has been no PSA progression in (i), when:
a. any serum PSA level measured during the period is not 25% or more higher than the serum PSA level of the patient measured prior to (i); or
b. any serum PSA level measured during the period is not 25% or more and not 2 ng/ml or more higher than the serum PSA level of the patient measured prior to step (i).

7. (canceled)

8. The method of claim 1, wherein there is a PSA progression in (ii)a, when:
a. a serum PSA level measured after the period is 25% or more higher than the lowest serum PSA level of the patient measured over the period of (i) or immediately prior to the administering of step (i); or
b. a serum PSA level measured after the period is 25% or more and 2 ng/ml or more higher than the lowest serum PSA level of the patient measured over the period of (i) or immediately prior to the administering of step (i).

9. (canceled)

10. The method of claim 1, wherein the first dose of the radioconjugate administered in step (i) is 100-400 μCi.

11.-15. (canceled)

16. The method of claim 1, where the second dose of the radioconjugate administered in step (ii) is 100-250 μCi.

17. (canceled)

18. The method of claim 1, wherein the patient has not received any dose of the radioconjugate prior to step (i), wherein the first dose of the radioconjugate administered in step (i) is 150 μCi, 200 μCi, or 250 μCi, and wherein the second dose of the radioconjugate administered in step (ii) is 100 or 150 μCi.

19. (canceled)

20. The method of claim 1, wherein the second dose of the radioconjugate administered in step (ii) is:
a. 150 μCi, if cumulative exposure to the radioconjugate for the patient prior to step (ii) is not higher than 450 μCi; or
b. 100 μCi, if cumulative exposure to the radioconjugate for the patient prior to step (ii) is 500 μCi.

21. (canceled)

22. The method of claim 1, wherein the second dose of the radioconjugate administered in step (ii) is lower than the dose of the radioconjugate administered in step (i).

23. The method of claim 1, further comprising (iii) administering to the patient a third dose of the radioconjugate after a second period of time after (ii), if the following conditions are met:
a. there is a PSA progression after the second period, relative to the lowest serum PSA level of the patient measured over the second period or immediately prior to the administering of (ii); and
b. the patient has a platelet count above 100×10E9 per liter of blood.

24. The method of claim 1, wherein cumulative exposure to the radioconjugate does not exceed 600 μCi.

25. The method of claim 1, wherein;
a. the antibody or an antigen binding fragment comprises:
i. a heavy chain complementarity determining region (HCDR) 1, a HCDR2 and a HCDR3 of a heavy chain variable region (VH) comprising the amino acid sequence of SEQ ID NO: 7 and a light chain complementarity determining region (LCDR) 1, a LCDR2, and a LCDR3 of a light chain variable region (VL) comprising the amino acid sequence of SEQ ID NO: 8, wherein the HCDR1, HCDR2, HCDR3, LCDR1, LCDR2 and LCDR3 are defined by the Kabat, Chothia, IMGT or AbM numbering system;
ii. a HCDR1, HCDR2, HCDR3, LCDR1, LCDR2 and LCDR3 having the amino acid sequences of SEQ ID NOs: 1, 2, 3, 4, 5, and 6, respectively;
iii. a VH and VL having the amino acid sequences at least 90% identical to SEQ ID NO: 7 and SEQ ID NO: 8, respectively; and/or
iv. a heavy chain sequence and a light chain sequence having the amino acid sequences at least 90% identical to SEQ ID NO: 11 and SEQ ID NO: 12,
respectively; and/or
b. the chelator is 1,4,7,10-tetraazacyclododecane-1,4,7,10-tetraacetic acid (DOTA); and/or
optionally:
c. the linker comprises or is a benzyl isothiocyanate, wherein the benzyl isothiocyanate optionally has (i) one or more alkyl substituents, (ii) one or more cyclic acid substituents, or (iii) substituents L1 and/or L2 at the benzylic position, wherein:
i. L1 is H or an alkyl or cyclic alkyl, when L1 is an alkyl or cyclic alkyl, the alkyl or cyclic alkyl can have carbon or heteroatom substitutions;
ii. L2 is H or an alkyl or cyclic alkyl, when L1 is an alkyl or cyclic alkyl, the alkyl or cyclic alkyl can have carbon or heteroatom substitutions; or
iii. L1 and L2 form a cycle.

26.-31. (canceled)

32. The method of claim 1, wherein the radioconjugate comprises the antibody conjugated to 2-(4-isothiocyanatobenzyl)-1,4,7,10-tetraazacyclododecane-1,4,7,10-tetraacetic acid with the chelated actinium-225 (²²⁵Ac), or 2-S-(4-isothiocyanatobenzyl)-1,4,7,10-tetraazacyclododecane-1,4,7,10-tetraacetic acid with the chelated actinium-225 (²²⁵Ac), wherein the antibody comprises a VH and VL having the amino acid sequences of SEQ ID NO: 7 and SEQ ID NO: 8, respectively, optionally, the antibody comprises a heavy chain sequence and a light chain sequence having the amino acid sequences of SEQ ID NO: 11 and SEQ ID NO: 12, respectively; the radioconjugate comprises an average of from about 1 to about 4 chelator molecules conjugated to the antibody; and/or provides a specific activity from about 50 μCi to about 350 μCi per about 2-3 mg of total antibody.

33.-36. (canceled)

37. The method of claim 1, wherein the radioconjugate is administered to the patient in a pharmaceutical composition comprising the radioconjugate, a radioprotectant, a surfactant and a buffer, with a pH of 5-6; optionally, the pharmaceutical composition further comprises a conjugate comprising the antibody or the antigen binding fragment conjugated to the chelator, optionally via the linker, without actinium-225 (²²⁵Ac) chelated by the chelator.

38. (canceled)

39. The method of claim 37, wherein the pharmaceutical composition is:
a. administered with a total antibody mass of 2 to 10 mg per dose;
b. administered at 12.5 μCi/mL, 25 μCi/mL, 37.5 Ci/mL or 50 μCi/mL (at the time of dosing); and/or
c administered to the patient intravenously.

40.-41. (canceled)

42. The method of claim 1, wherein the patient:
a) has a prior treatment with at least one androgen receptor pathway inhibitor (ARPI);
b) can have a prior chemotherapy treatment;
c) has not had prior radioconjugate therapy;
d) has no prior radiotherapy with >30% red marrow distribution;
e) has received 0 to 12 prior lines of therapy; and/or
f) has met one or more of the following criteria:
1) mCRPC with histologic confirmation of adenocarcinoma;
2) prior exposure to at least one androgen receptor-targeted therapy, or prior taxane or other chemotherapy;
3) treatment with other agents for prostate cancer, if received, discontinued ≥2 weeks prior to first dose of the radioconjugate;
4) prior orchiectomy or medical castration; or, for patients who have not undergone orchiectomy, receiving ongoing androgen deprivation therapy with a gonadotropin releasing hormone (GnRH) agonist or antagonist prior to the first dose of the radioconjugate;
5) palliative radiotherapy completed >2 weeks prior to the first dose of the radioconjugate, except for palliative radiotherapy for pain, which may be used any time prior to first dose;
6) Eastern Cooperative Oncology Group (ECOG) performance status of 0 or 1;
7) hematology laboratory parameters within the following range:
a. hemoglobin >9.0 g/dL
b. absolute neutrophil count >1.5×10⁹/L
c. platelet count >100×10⁹/L
8) clinical chemistry laboratory parameters within the following range before the first dose of study drug:
a. serum total bilirubin <1.5×upper limit of normal (ULN)
b. aspartate aminotransferase (AST) and alanine aminotransferase (ALT) ≤2.5×ULN (or ≤4×ULN for participants with tumor involvement in the liver)
c. calculated or measured creatinine clearance >50 mL/min/1.73 m² as determined by Modification of Diet in Renal Disease formula
d. Grade ≤1 proteinuria,
g) does not have one or more of the following:
1) prior treatment with radium Xofigo® (Ra 223 dichloride), strontium, or samarium therapy or radioconjugate therapy;
2) prior radiation therapy encompassing >30% of expected red marrow distribution;
3) diffuse bone or bone-marrow involvement);
4) spinal cord compression (unless treated, stable, and approved by the sponsor medical monitor);
5) active CNS metastases;
6) known history of myelodysplastic syndrome, leukemia, or hematological malignancy with features suggestive of myelodysplastic syndrome/acute myeloid leukemia at any timepoint;
7) toxicity from prior anticancer therapy has not resolved to baseline levels or to Grade ≤1 (except alopecia, radiation tissue fibrosis, or peripheral neuropathy);
8) malignancy diagnosis other than the disease under study within 2 years prior to the first dose of study drug, except for squamous and basal cell carcinoma of the skin, non-muscle invasive bladder cancer, or any malignancy considered cured or has minimal risk of recurrence within 1 year of first dose of study drug (in the opinion of both the investigator and sponsor's medical monitor); or
9) any systemic anti-neoplastic therapy ≤30 days prior to the first dose of the radioconjugate except for luteinizing hormone-releasing hormone agonists/antagonists or GnRH agonists/antagonists, and androgen axis drugs must have been discontinued ≥2 weeks prior to the first dose of the radioconjugate.

43.-45. (canceled)

46. The method of claim 1, wherein the treatment:
a. provides a lower rate of severe toxicities (toxicity of Grade 3 or higher according to the NCI CTCAE version 5.0) compared to the administering of the first dose of (i) at a fixed dose level and at a fixed dose schedule (e.g., compared to the administering of the first dose of (i) once every 8 weeks); and/or
b. results in effective treatment of the prostate cancer, as measured by the PSA response, and/or Overall response rate (ORR) according to response criteria of Prostate Cancer Working Group 3 (PCWG3).

47. (canceled)

48. The method of claim 1, wherein the prostate cancer is advanced prostate cancer, locally advanced prostate cancer, metastatic castration-sensitive prostate cancer (CSPC), metastatic castration-resistant prostate cancer (CRPC), non-metastatic CSPC, non-metastatic CRPC, or metastatic prostate cancer.

49. The method of claim 1, wherein the prostate cancer is metastatic hormone sensitive prostate cancer (mHSPC), optionally wherein the mHSPC is mHSPC with:
i. non-castrate levels of testosterone (testosterone >150 ng/dL),
ii. no evidence of metastasis to visceral organs;
iii. metastasis in no more than five locations in the patient's body; and/or
iv. metastasis in no more than five locations in the patients' body and with no evidence of metastasis to visceral organs.

50. (canceled)

51. The method of claim 49, wherein the patient does not have undergone orchiectomy and is not receiving ongoing androgen deprivation therapy with a gonadotropin releasing hormone (GnRH) analog, agonist, or antagonist prior to the first dose of the radioconjugate.

52.-55. (canceled)

56. The method of claim 49, wherein the patient has not received any dose of the radioconjugate prior to step (i), wherein the first dose of the radioconjugate administered in step (i) is 150 μCi or 200 μCi, and wherein the second dose of the radioconjugate administered in step (ii) is 100 or 150 μCi.

57.-59. (canceled)
